(19) United States
(12) Patent Application Publication
(10) Pub. No.: US 2021/0220272 A1
(43) Pub. Date: Jul. 22, 2021

(54) BIPHASIX CANNABINOID DELIVERY

(71) Applicant: Altum Pharmaceuticals Inc., Vancouver (CA)

(72) Inventors: Ahmad Doroudian, Vancouver (CA); Patrick Frankham, Rosemere (CA)

(21) Appl. No.: 17/000,829

(22) Filed: Aug. 24, 2020

Related U.S. Application Data
(63) Continuation of application No. 15/990,072, filed on May 25, 2018, now abandoned.
(60) Provisional application No. 62/511,686, filed on May 26, 2017.

(51) Int. Cl. A61K 9/127; A61K 47/02; A61K 47/36; A61K 9/06; A61K 45/06; A61K 31/485; A61K 31/4468; A61K 9/00; A61K 47/18; A61K 47/10; A61K 47/28; A61K 47/44; A61K 47/06; A61K 47/20; A61K 31/047; A61K 9/107; A61K 31/05

(52) U.S. Cl. CPC A61K 9/127; A61K 31/05; A61K 47/36; A61K 9/06; A61K 45/06; A61K 31/485; A61K 31/4468; A61K 9/0014; A61K 9/1277; A61K 47/186; A61K 47/10; A61K 47/28; A61K 47/183; A61K 47/44; A61K 47/06; A61K 47/20; A61K 31/047; A61K 9/1075; A61K 47/02

(57) ABSTRACT

A biphasix multilayered lipid vesicle cannabinoid composition comprising: (a) a first phase comprising a first oil-in-water emulsion; and (b) a second phase suspended in the first phase, the second phase comprising multilamellar lipid vesicles, the multilamellar lipid vesicles entrapping a second oil-in-water emulsion, wherein at least one of the first and second oil-in-water emulsions comprises a therapeutically effective amount of a cannabinoid. The composition for transdermal and topical administration for the treatment of pain.

### CROSS-REFERENCE TO RELATED APPLICATIONS

This application is a continuation of and claims the benefit and priority to U.S. patent application Ser. 15/990,072, filed May 25, 2018, which claims the benefit of priority to U.S. Provisional Application Ser. No. 62/511,686, filed May 26, 2017. The entire contents of the above referenced applications are hereby expressly incorporated by reference in their entireties. Any and all priority claims identified in the Application Data Sheet, or any correction thereto, are hereby incorporated by reference under 37 CFR 1.57.

### FIELD OF THE INVENTION

The invention relates to compositions and formulations for cannabinoid delivery and related methods and uses. More particularly, the present invention relates to cannabinoid biphasix multilayered lipid vesicle (MLV) compositions and formulations, methods of making the compositions and formulations, methods of treating pain associated with dermatological and other conditions with the cannabinoid biphasix multilayered lipid vesicle (MLV) compositions and formulations, and methods for dermatological delivery of the cannabinoid biphasix (MLV) compositions and formulations. The cannabinoid biphasix multilayered lipid vesicles may be formulated into a variety of formats for topical and mucosal administration.

### BACKGROUND OF THE INVENTION

Cannabis sativa, commonly known as marijuana, and its major psychoactive ingredient, Δ⁹-tetrahydrocannabinol (Δ⁹-THC), and various other Cannabis constituents, termed cannabinoids, have been widely studied. Herbal Cannabis contains more than 400 chemicals and over 60 cannabinoids, including the tetrahydrocannabinols (THC), Δ⁹-THC, ⁹-THC Propyl Analogue (THC-V); Cannabidiol (CBD); Cannabidiol Propyl Analogue (CBD-V); Cannabinol (CBN), Cannabichromene (CBC); cannabinodiol (CBDL); cannabicyclol (CBL); Cannabichromene Propyl Analogue (CBC-V); cannabielsoin (CBE); cannabitriol (CBT) and Cannabigerol (CBG). Herbal Cannabis also includes more than a dozen terpenoids and several flavonoids.

“Cannabinoid receptors” are cells in the brain and other organs that contain specific protein receptors which recognize THC and some other cannabinoids and trigger cell responses. Some of the cannabinoids do not bind to these cannabinoid receptors and exert their effects by other ways. CB1 receptors are found in high concentrations within the brain and spinal cord. They are also present in certain peripheral cells and tissues (some neurons, some endocrine glands, leukocytes, spleen, heart and parts of the reproductive, urinary and gastrointestinal tracts). CB2 receptors are expressed primarily by immune cells and tissues (leukocytes, spleen and tonsils).

Cannabinoids are used therapeutically in the treatment of variety of disorders and discomforts as they have effects on cannabinoid receptors or related structures and/or mechanisms.

Cannabinoids are lipophilic and potentially acid-labile compounds. Because of their hydrophobic nature, cannabinoids are poorly absorbed systemically from oral dosage forms because of the poor dissolution of cannabinoids in the aqueous environment of gastrointestinal tract. Because of their poor absorption and poor bioavailability, oral formulations are disadvantageous.

While the skin may be a desirable target, even lipophilic and low molecular weight compounds generally may only transfer in small amounts across the skin, resulting in difficulty in achieving therapeutic levels of drug in the bloodstream. Topical formulations may provide better patient compliance versus injections or intravenous administration, however, depending on the type of formulation, the release of the cannabinoid may vary and thus formulation effectiveness may vary. Cannabinoids have been formulated into topical compositions as described for example in U.S. 2012/0264818, U.S. 2013/0274321, U.S. 2016/094810, U.S. Pat. Nos. 9,095,563 and 9,375,417.

Liposomal compositions for delivery of hydrophilic biologics such as interferon are for use in the treatment of cervical dysplasia are described in U.S. Pat. No. 6,656,499, WO 2015/023600, WO 2015/023601, and WO 2008/119160. It would be advantageous to develop a liposomal based composition comprising a lipophilic active, i.e. cannabinoid, that is stable and able to deliver cannabinoids in a safe manner that is not irritating to the skin or mucosa or damaging to the skin or mucosa with repeated use. It is desired that such composition delivers desired amounts of cannabinoids as needed to a desired area in a controlled manner for rapid and/or slow release to achieve a therapeutically effective dose to treat pain associated with different types of conditions, for example dermatological and related conditions. Liposomal based cannabinoid compositions may help to avoid the issue of addiction associated with opioid pain treatment.

The present invention is directed toward overcoming one or more of the problems discussed above.

### SUMMARY OF THE INVENTION

Controlled delivery coupled with more effective release, is beneficial for the treatment of pain. Topical and transdermal application of cannabinoids targets the areas of pain anywhere on a body and provides for easy repeated use as needed. The novel release platform for cannabinoids, cannabinoid biphasix multilayered lipid vesicle (MVL) composition of the invention, helps to achieve these aspects.

The invention presented herein in aspects demonstrates a cannabinoid-containing biphasix multilayered lipid vesicle (MLV) composition. The MVL are comprised of lipid bilayers that entrap both aqueous and oil phases in the form of a stabilized emulsion. Cannabinoids are lipophilic and get entrapped in the oil phase of the submicron emulsion and may be further entrapped between the phospholipid bilayers. This achieves enhanced formulation performance compared to traditional creams, gels or ointments including conventional liposomes.

Topical delivery or transdermal delivery of the cannabinoid-containing composition may decrease pain in general, and pain associated with medical conditions without inducing abnormal behavior or other adverse effects. The compositions of the invention have use for the treatment of any type of pain inclusive of pain associated with a wide variety of dermatological conditions. Treatment for eye pain is also within the scope of the invention.

In an embodiment, the present invention provides a pharmaceutically effective amount of biphasix multilayered lipid vesicle (MLV) composition which comprises a pharmaceutically effective amount of a cannabinoid for topical or transdermal delivery of the cannabinoid to skin, mucous membrane, or eye of a user.

In an embodiment, the present invention comprises a formulation comprising a pharmaceutically effective amount of biphasix multilayered lipid vesicle (MLV) composition for topical or transdermal delivery of the cannabinoid to skin, mucous membrane, or eye of a user.

In aspects, the composition is provided as suspended droplets of cannabinoid within at least one lipid bilayer. In further aspects, cannabinoid is further entrapped between the lipid bilayer itself.

In aspects, the composition is a biphasix multilayered multilayered lipid vesicle (MLV) composition comprising one or more cannabinoids. In aspects such composition can be formulated in a variety of formulation formats including but not limited to: cream, lotion, liquid, gel, foam, drops, suppository, ointments, shampoo, soap bar, sprays and patches. The Cannabis composition or formulations containing such compositions can be provided packaged in an amount containing a number of doses, labeled with instructions for use or in a kit for use with instructions. The composition can be so formulated to have desirable and in aspects, improved organoleptic properties for use.

The biphasix multilayered lipid vesicle composition of the invention is a liposome-based technology designed to enable cannabinoid molecules to be delivered on and into the skin, mucosal membranes and the eye. The biphasix multilayered lipid vesicle composition comprises phospholipid vesicles which are multi-lamellar (multi-compartmental) structures in aspects with up to 20 layers, or up to 15 layers or between about 15 and 20 layers separated by an oil-in-water microemulsion.

The cannabinoid is formulated as suspended droplets within a stabilized microemulsion surrounded by one or more lipid bilayers (droplets contained in a core). The lipid bilayers each separated by microemulsion compartments. As cannabinoids are lipophilic, they may also incorporate in between the phospholipid bilayers themselves. Thus each separate lipid bilayer compartment may be separated by microemulsions (aqueous/oil droplet) that contains the cannabinoid lipid droplets. The cannabinoid droplets can be formulated with a surrounding surfactant if desired. Furthermore, the cannabinoid droplets can consist of cannabinoid or comprise cannabinoid, meaning that a further agent may be incorporated into the droplet. The further agent may be a further therapeutic agent if desired. Optional stabilizers and/or optional anti-aggregants may be incorporated with the phase containing the cannabinoid droplets. Stabilizers such as glycerin and water soluble esterified Vitamin E, d-α-Tocopheryl polyethylene glycol 10 succinate (TPGS or Vitamin E TPGS) can be utilized to stabilize chemically the formulation. These are added after melting the phospholipid and using it as a net to trap the water CBD emulsion.

In this manner, cannabinoid can be provided within multiple structures of the MLV providing for immediate and further longer lasting delivery of the cannabinoid.

In one aspect, a biphasix multilayered lipid vesicle composition comprises a suspension of lipid-bilayer vesicles having entrapped therein, an oil-in-water emulsion, one or more cannabinoid compounds, analogues, and/or cannabinoid agonists. The composition may optionally comprise an antioxidant and/or anti-aggregant. In aspects the antioxidant is provided in an amount between about 0.01 to about 0.5 weight percent and may be methionine, in aspects L-methionine. In aspects, the anti-aggregant is present in an amount of about 0.1 to about 5 mg/kg, in aspects is a pharmaceutically acceptable salt of arginine L-arginine hydrochloride.

According to an aspect of the invention is a biphasix multilayered lipid vesicle composition comprising: (a) a first phase comprising an oil-in-water emulsion which itself comprises oil, water, cannabinoid; and (b) a second phase comprising multilamellar lipid vesicles suspended in said first phase wherein said vesicles contain entrapped therein a composition comprising an oil-in-water emulsion which itself comprises oil, water, and cannabinoid, wherein each phase optionally comprises an amount sufficient of a stabilizer to stabilize the cannabinoid against oxidation, further wherein said composition comprises a therapeutically effective amount of said cannabinoid.

In aspects, the cannabinoid composition is formulated with a base cream in a variety of ratios to provide a desired amount of cannabinoid active and to make a formulation with desired organoleptic properties and consistency for dermal and mucosal applications to help treat and/or alleviate pain. Formulations are suitable for continued use and multiple applications with minimal to no dermal or mucosal irritation or damage. Formulations may take the form of a cream, lotion, liquid, liquid spray, gel, foam, drops, suppository, ointment or patch. The formulations may comprise any desired amount of active cannabinoid, in aspects up to 10% by weight cannabinoid, in aspects up to 19% by weight, up to 18% by weight, up to 17% y weight, up to 16% by weight, up to 15% by weight, up to 14% by weight, up to 13% by weight, up to 12% by weight, up to 11% by weight, up to 10% by weight, up to 9% by weight, up to 8% by weight, up to 7% by weight, up to 6% by weight, up to 5% by weight, up to 4% by weight, up to 3% by weight, up to 2% by weight and up to 1% by weight, each amount being either in the composition or the formulation.

According to an aspect of the invention is a method for the treatment of pain comprising the administration of a biphasix multilayered lipid vesicle cannabinoid composition or a formulation comprising the composition. In aspects the cannabinoid is CBD and in aspects is present in an amount of up to about 1% by weight of the composition/formulation.

According to another aspect of the invention is a method for treating pain or pain symptoms in a patient, which method comprises administering to the dermis or mucosa of a patient a therapeutically effective amount of a biphasix multilayered lipid vesicle cannabinoid composition comprising; a) a first phase comprising an oil-in-water emulsion which itself comprises oil in water, wherein a sufficient amount of oil is employed to form a composition suitable for topical application, and wherein the water comprises optional antioxidant and an optional anti-aggregant; and (b) a second phase comprising multilamellar lipid vesicles suspended in said first phase wherein said vesicles contain entrapped therein a composition comprising an oil-in-water emulsion wherein the water phase comprises, an optional antioxidant and an optional anti-aggregant, wherein the composition comprises a therapeutically effective amount of said cannabinoid. In aspects, the cannabinoid is incorporated into the oil phase of the submicron emulsion. In aspects, at least one of the first and second oil-in-water emulsion is comprised of oil droplets having a size of from about 0.1 μm to about 1 μm.

In another aspect, the invention is drawn to a method of treating dermal pain in a subject by the transdermal administration of the cannabinoid biphasix multilayered lipid vesicle composition or formulation comprising the composition to the dermis (skin) of the subject. The application can be repeated as required several times during the day and used daily if required. Absorption may be increased by raising the skin temperature by vigorous rubbing of the composition or formulation onto the dermis (skin) and by forcing some cream to penetrate thru the skin pores of the epidermis.

In another aspect, the invention is drawn to a method of treating pain in a subject by the topical administration of the cannabinoid biphasix multilayered lipid vesicle composition or formulation containing such composition to skin of a subject.

In another aspect, the invention is drawn to a method of treating pain in a subject by the transdermal administration of the cannabinoid biphasix multilayered lipid vesicle composition or formulation containing such composition to skin of a subject.

In another aspect, the invention is drawn to a method of treating pain in a subject by the mucosal administration of the cannabinoid biphasix multilayered lipid vesicle composition or formulation containing such composition to the subject. Mucosal administration can be to mucosa of the nose, mouth, vagina or rectum.

In another aspect, the invention is drawn to a method of treating pain in a subject by the topical administration of the cannabinoid biphasix multilayered lipid vesicle composition or formulation containing such composition to an eye of the subject.

Topical administration is to an area of pain on skin. The skin may healthy and not compromised and thus the composition may be administered repeatedly to said skin. Administration may be by vigorously massaging into the skin to raise the skin temperature such that the composition may penetrate pores in the epidermis of the skin.

Topical administration may be to compromised skin, that comprises damage to the stratum corneum. Compromised skin may be physically compromised by cuts, scrapes, wounds, bites, incisions, blisters and/or punctures. In this aspect, topical administration helps to alleviate pain during healing of the compromised skin. Compromised skin may also be due to a dermatological condition that comprises inflammation and is selected from acne, hives, psoriasis, heat burns, sunburn, chemical burns, dermatitis, keratosis, rosacea, carbuncle, eczema, cellulitis, measles, lupus or impetigo. Topical administration helps to alleviate pain, irritation and inflammation of the dermatological condition.

Administration of the cannabinoid biphasix MLV composition can be repeated multiple times a day, once a day, continual daily use. The composition can be freely used to help alleviate pain.

The compositions of the invention comprises one or more cannabinoids and may be selected from (THC), Δ⁹-THC, ⁹-THC Propyl Analogue (THC-V); Cannabidiol (CBD); Cannabidiol Propyl Analogue (CBD-V); Cannabinol (CBN), Cannabichromene (CBC); cannabinodiol (CBDL); cannabicyclol (CBL); Cannabichromene Propyl Analogue (CBC-V); cannabielsoin (CBE); cannabitriol (CBT), Cannabigerol (CBG), pharmaceutically acceptable salts of these cannabinoids, cannabinoid prodrugs, cannabinoid agonists, synthetic analogs thereof and any combination of the aforementioned.

In aspects embodiments, the cannabinoid is a cannabinol, CBN; or a cannabidiol, CBD. THC can also be used as desired as are cannabinoid analogues and mixtures thereof. In aspects, the cannabinoids or cannabinoid analogues are selected from the group consisting of cannabinol, cannabidiol, Δ⁹-tetrahydrocannabinol, Δ⁸-tetrahydrocannabinol, 11-hydroxy-tetrahydrocannabinol, 11-hydroxy-Δ⁹-tetrahydrocannabinol, levonantradol, Δ¹¹-tetrahydrocannabinol, tetrahydrocannabivarin, dronabinol, amandamide, nabilone, a combination thereof, a natural or synthetic analogue thereof, and a natural or synthetic molecule with a basic cannabinoid structure. Mixtures of two or more cannabinoids may also be used; for example, CBD and THC may be used in a 1:1 ratio or any ratio as desired.

In aspects the biphasix multilayered lipid vesicle composition comprises a cannabinoid in non-limiting amounts, in aspects the cannabinoid is CBD. The CBD may be provided in a variety of formats such as wax, oil or powdered CBD that comprises or essentially consists of CBD/starch, in aspects the starch is maltodextrin. The invention has in aspects but is not limited to:
1. A biphasix multilayered lipid vesicle cannabinoid composition comprising:

(a) a first phase comprising a first oil-in-water emulsion; and

(b) a second phase suspended in the first phase, the second phase comprising multilamellar lipid vesicles, the multilamellar lipid vesicles entrapping a second oil-in-water emulsion,

wherein at least one of the first and second oil-in-water emulsions comprises a therapeutically effective amount of a Cannabis-derived compound.
2. The cannabinoid composition of claim 1, wherein the first and second oil-water-emulsions are the same or different.
3. The cannabinoid composition of claim 1 or 2, wherein the Cannabis-derived compound is a cannabinoid.
4. The cannabinoid composition of any one of claims 1 to 3, wherein the Cannabis-derived compound is a cannabinoid selected from the group consisting of natural or synthetic cannabinoid, tetrahydrocannabinols (THC), Δ⁹-THC, ⁹-THC Propyl Analogue (THC-V); Cannabidiol (CBD); Cannabidiol Propyl Analogue (CBD-V); Cannabinol (CBN), Cannabichromene (CBC); cannabinodiol (CBDL); cannabicyclol (CBL); Cannabichromene Propyl Analogue (CBC-V); cannabielsoin (CBE); cannabitriol (CBT), Cannabigerol (CBG), pharmaceutically acceptable salts of these cannabinoids, cannabinoid prodrugs, cannabinoid agonists, synthetic analogs thereof and combinations thereof.
5. The cannabinoid composition of claim 4, wherein the cannabinoid is CBD.
6. The cannabinoid composition of claim 5, wherein CBD is present in an amount of up to about 10% by weight.
7. The cannabinoid composition of any one of claims 1 to 5, further comprising an anti-oxidant in (a) and/or (b).
8. The cannabinoid composition of any one of claims 1 to 7, wherein at least one of the first and second oil-in-water emulsion is comprised of oil droplets having a size of from about 0.1 μm to about 1 μm.
9. The cannabinoid composition of any one of claims 1 to 8, wherein at least 30% of said Cannabis-derived compound is entrapped within said vesicles.
10. The cannabinoid composition of any one of claims 1 to 9, wherein Cannabis-derived compound is further entrapped between phospholipid bilayers of the multilamellar vesicles.
11. The cannabinoid composition of any one of claims 1 to 10, wherein said multilamellar vesicles comprise from about 2 to about 4 by weight percent cholesterol.
12. The cannabinoid composition of any one of claims 1 to 11 formulated as a cream, lotion, liquid, gel, foam, drops, suppository, ointment, spray or patch.
13. The cannabinoid composition of claim 12, wherein said formulation comprises up to 5% by weight CBD, up to 4% CBD, up to 3% by weight CBD, up to 2% by weight CBD or up to 1% by weight CBD.
13a. The cannabinoid composition of any one of claims 1 to 13, further comprising morphine, fentanyl, oxycodone or codeine.
14. A method for the treatment of pain in a subject comprising transdermally administrating the cannabinoid composition of any one of claims 1 to 13a to an area of pain on skin.
15. The method of claim 14, wherein the skin is healthy and not compromised.
16. The method of claim 15, wherein said composition may be administered repeatedly to said skin.
17. The method of claim 14, 15 or 16, wherein said cannabinoid composition is vigorously massaged into the skin to raise the skin temperature such that the composition may penetrate pores in the epidermis of the skin.
18. A method for the treatment of pain in a subject comprising topically administering the cannabinoid composition of any one of claims 1 to 13a to compromised skin.
19. The method of claim 18, wherein said compromised skin comprises damage to the stratum corneum.
20. The method of claim 18 or 19, wherein said skin is physically compromised by cuts, scrapes, wounds, bites, incisions, blisters and/or punctures.
21. The method of claim 18 or 19, wherein said topical administration helps to alleviate pain during healing of said skin.
21. The method of claim 18, wherein the compromised skin is due to a dermatological condition.
22. The method of claim 21, wherein the dermatological condition comprises inflammation and is selected from acne, hives, psoriasis, heat burns, sunburn, chemical burns, dermatitis, keratosis, rosacea, carbuncle, eczema, cellulitis, measles, lupus or impetigo.
23. The method of claim 21 or 22, wherein said topical administration helps to alleviate pain, irritation and inflammation of said dermatological condition.
24. The method of any one of claims 18 to 23, wherein said composition may be topically administered repeatedly to said skin.
25. A method for the treatment of pain in a subject comprising topically administering the Cannabis composition of any one of claims 1 to 13a to mucosa of the mouth, nose, vagina or rectum.
26. The method of claim 25, wherein said Cannabis composition relieves pain and irritation.
27. The method of claim 25 or 26, wherein said Cannabis composition may be administered repeatedly to said skin.
28. The composition of any one of claims 1 to 13a, formulated as eye drops, optionally comprising a lubricant, redness reliever, astringent, and/or antibiotic.
29. The composition of any one of claims 1 to 13a, further comprising salicylic acid, oxyacetic acid, salicylates, propionic acid derivatives, acetic acid derivatives, enolic acid derivatives, fenamic acid derivatives, coxibs, sulphonanilides and mixtures thereof.
30. The composition of claim 29, further comprising an antibiotic selected from the group consisting of chloramphenicol, fusidic acid, fluoroquinolones, aminoglycoside, polymycin B sulfate and mixtures thereof.
31. The composition of any one of claims 1 to 13a, formulated as a cream comprising up to 5% by weight CBD and a base cream formulation.
32. The composition of claim 31, wherein said CBD is provided in an amount of up to about 1% by weight, up to about 2% by weight, up to about 3% by weight or up to about 4% by weight.
33. The composition of claim 32, wherein said cream formulation comprises at least two ingredients selected from the group consisting of water, ceteraryl octanoate, glycerin, shea butter, sweet almond oil, palm oil, jojoba oil, aloe barbaensis, maris sal, potassium sorbate, sclerotium gum, xanthum gum, tocopheryl acetate, camellia sinensis leaf extract, corral powder and any combination thereof.
34. A cannabinoid composition for topical administration to the skin or mucosa, the cannabinoid composition comprising biphasix multilayered lipid vesicles, the cannabinoid composition comprising:

(a) a first phase comprising an oil-in-water emulsion which itself comprises oil, water, and a cannabinoid; and

(b) a second phase comprising multilamellar lipid vesicles suspended in said first phase wherein said vesicles contain entrapped therein a composition comprising an oil-in-water emulsion which itself comprises oil, water and cannabinoid, and wherein cannabinoid may be further entrapped in lipid bilayers of said vesicles;

wherein said composition comprises a therapeutically effective amount of cannabinoid for alleviating pain.
35. The cannabinoid composition of claim 34, wherein said cannabinoid is CBD.
36. The cannabinoid composition of claim 34 or 35, wherein said composition penetrates the epidermal layer of compromised skin.
37. The cannabinoid composition of claim 34 or 35, wherein said composition penetrates the mucosa.
38. The cannabinoid composition of any one of claims 34 to 37, comprising up to about 10% by weight CBD.
39. A cannabinoid formulation comprising the cannabinoid composition of any one of claims 1 to 11 or 34 to 38 for the treatment of pain, the formulation provided as a cream, lotion, liquid, gel, foam, drops, suppository, ointment, spray or patch.
40. The cannabinoid formulation of claim 39, provided as a cream comprising up to about 1% by weight CBD.
41. The cannabinoid formulation of claim 40, wherein said cream comprises at least two ingredients selected from the group consisting of water, ceteraryl octanoate, glycerin, shea butter, sweet almond oil, palm oil, jojoba oil, aloe barbaensis, maris sal, potassium sorbate, sclerotium gum, xanthum gum, tocopheryl acetate, camellia sinensis leaf extract, corral powder and any combination thereof.
42. A multicompartmental lipid vesicle composition suitable for topical or transdermal administration to skin or mucosa, said composition comprising a cannabinoid, wherein said lipid vesicles each comprise aqueous compartments, bilayer compartments, micellar compartments and oil compartments, wherein said cannabinoid is present in at least two of said compartments.
43. The composition of claim 42, wherein said cannabinoid is present in three of said compartments.
44. The composition of claim 42, wherein said cannabinoid is present in all of said compartments.
45. The composition of any one of claims 42 to 44, wherein said cannabinoid is CBD.
46. The composition of any one of claims 42 to 45, wherein upon application to said skin or mucosa, said cannabinoid is released rapidly followed by controlled slow release to alleviate pain.
47. A method of delivering a cannabinoid transdermally or topically to a subject to alleviate pain, the method comprising the steps of:

a) providing the composition of any one of claims 1-13a, 28-38 or 42-46;

b) providing a backing layer selected from the group consisting of a patch, strip, bandage and covering, for holding said composition;

c) placing an effective amount of said composition onto said backing layer; and,

d) attaching said backing layer to the skin of said person so that said composition is in contact with said skin.
48. The method of claim 47 comprising the additional steps of:

e) providing an adhesive mixture containing an effective amount of said composition; and,

f) carrying out step c) by applying said adhesive mixture onto said backing layer.
49. The method of claim 48, comprising the additional step of providing a reservoir means to said backing layer for holding said composition.
50. The method of claim 49, wherein said reservoir means is any one or combination of a member of the group consisting of a cavity, matrix material, adhesive layer and film.
51. The method of claim 47 wherein after step d), said composition is maintained in contact with said skin for an effective period of time to alleviate, reduce pain.
52. A structure for administering Cannabis to skin, comprising:

at least one layer of backing material suitable for attachment to said skin; and,

the composition of any one of claims 1 to 13 or 28 to 38.
53. The structure of claim 52, wherein said backing material is any one or combination of a member selected from the group consisting of fabric, plastic, metal foil, rubber, resin film and membrane.
54. The structure of claim 53 wherein said structure further comprises a reservoir means that includes a rate control means for regulating the flow of said composition to said skin.
55. A composition for pain, the composition comprising CBD, Gelucire 44/14, Cremer Miglycol 810, Gelucire 50/13, butylated hydroxytoluene, kolliphor EL, Phospholipon 90H, Vit E TPGS, propylene glycol and carnosic oil 1% in MCT.
56. The composition of claim 55, further comprising two or more of: water, Ceteraryl octanoate, Glycerin, Shea butter, Sweet almond oil, Palm oil, Jojoba oil, aloe barbaensis, Maris sal, Potassium sorbate, Sclerotium gum, Xanthan gum, Tocopheryl acetate, Camellia sinensis leaf extract and Corral powder.
57. A concentrated biphasic cannabinoid composition comprising powdered CBD, GELUCIRE 44/14, CREMER Miglyol 810, Butylated Hydroxytoluene, Benzalkonium Chloride 50% Solution, Propylparaben, Sodium phosphate, Dibasic, Heptahydrate, Sodium phosphate Monobasic, anhydrous Edetate Disodium Dihydrate, Kolliphor EL, Phospholipon 90H, Cholesterol, Vit E TPGS, Propylene Glycol and Purified Water Q.S.
58. The concentrated biphasic cannabinoid composition of claim 57, admixed with a cream base in a ratio of about 1:9 or about 2:8.
59. A method to make a cannabinoid biphasix multilayered lipid vesicle composition comprising:

a) preparing a lipophilic cannabinoid-in-water emulsion,

b) preparing oil and/or solid/semisolid lipophilic ingredients and cannabinoid,

c) homogenize a) and b) for a period of time to obtain relatively small droplet size,

d) prepare and heat a lipid phase melt (anhydrous plastic proliposome gel), and

e) the cannabinoid-in-water emulsion of a) is added to c) and d) and vigorously mixed to make the cannabinoid composition.
60. Use of the composition or formulation of any one of claims 1-13a, 29-46 or 55-58 for the treatment of pain.
61. The use of claim 60, wherein said use is topical.
62. The use of claim 59, 60 or 61 wherein said use is to skin or mucosa.

While the aforementioned aspects recite “comprising”, we submit that this transitional phrase could be replaced by “consisting essentially of” or “consisting of” in any of these aspects.

These and other objects and features of the invention will be more fully appreciated when the following detailed description of the invention is read in conjunction with the accompanying drawings.

### BRIEF DESCRIPTION OF THE DRAWINGS

FIG. 1 represents the structure, assembly and properties of biphasic MVL of the invention for carrying a cannabinoid;

FIG. 2 is a scanned image, magnified 440× of vesicles made for use as a topical lotion;

FIG. 3A is a scanned image of multilamellar liposomes prepared using an “anhydrous plastic proliposome-gel” (‘melt’ or ‘fusion’) method.

FIG. 3B is a scanned image of multilamellar liposomes, the same composition as in 2A, but prepared by a solvent evaporation method.

FIG. 4 shows a particle size distribution pattern of biphasix placebo formulation that does not contain cannabinoid;

FIG. 5 is an optical microscope image of biphasix placebo oil-in-water emulsion;

FIG. 6 is a ×200 magnification of FIG. 5; and

FIG. 7 shows CBD absorption in intact and stripped skins.

### DESCRIPTION OF THE INVENTION

Unless otherwise explained, all technical and scientific terms used herein have the same meaning as commonly understood by one of ordinary skill in the art to which this disclosure belongs.

It is also to be understood that the terminology used herein is for the purpose of describing particular aspects only, and is not intended to be limiting.

In understanding the scope of the present application, the articles “a”, “an”, “the”, and “said” are intended to mean that there are one or more of the elements.

Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. In aspects, the composition does not comprise an interferon. In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms may refer to a measurable value such as an amount, a temporal duration, and the like, is meant to encompass variations of ±20% or ±10%, more typically ±5%, even more typically ±1%, and still more typically ±0.1% from the specified value, as such variations are appropriate to perform the disclosed methods.

Compositions of the present invention comprise one or more cannabinoids. By “cannabinoids” is meant a class of diverse chemical compounds that act on cannabinoid receptors on cells that affect neurotransmitter release in the brain. The Cannabis plant produces an estimated 80+ cannabinoids, each of which has unique pharmacologic effects. Δ⁹-tetrahydrocannabinol (Δ⁹-THC), is the primary psychoactive compound of Cannabis. Cannabisrefers to various strains of plants Cannabis sativa or Cannabis indica. Generally, cannabinoids are collected from the female plant. Thus “cannabinoid” is included herein, tetrahydrocannabinols (THC), Δ⁹-THC, ⁹-THC Propyl Analogue (THC-V); Cannabidiol (CBD); Cannabidiol Propyl Analogue (CBD-V); Cannabinol (CBN), Cannabichromene (CBC); cannabinodiol (CBDL); cannabicyclol (CBL); Cannabichromene Propyl Analogue (CBC-V); cannabielsoin (CBE); cannabitriol (CBT), Cannabigerol (CBG), pharmaceutically acceptable salts of these cannabinoids, cannabinoid prodrugs, cannabinoid agonists, synthetic analogs thereof and any combination of the aforementioned. Cannabinoids to use in the present invention also include the carboxylic acid forms of cannabinoids, or the cannabinoid acids.

Cannabinoids to use in the present invention include any of the cannabinoids as discussed above. In one embodiment, the cannabinoid to use in the composition is CBN, CBDα, CBD, THC, THCα, or mixtures of CBD (or CBDα) or CBN and THC (or THCα). Mixtures of CBD, CBN or CBDα and THC or THCα can be, for example, 1:1 w/w or any other mixture. Various ratios of the above-described cannabinoids can be used for the topical applications described herein. The ratios can be adjusted based on pharmacological effects required. Ratios of enriched/purified cannabinoids for the cannabinoid products of the invention can be adjusted, such as, for example, 1:1 w/w CBD:THC. Ratios include but are not limited to 0.1:1, 0.2:1, 0.3:1, 0.4:1, 0.5:1, 0.6:1, 0.7:1, 0.8:1, 0.9:1, 1:1, 1:1.2, 1:1.5, 1:1.3, 1:1.5, 1:1.7, 1:2, 1:3, 1:4, 1:5, 1:6, 1:7, 1:8 or 1:10 (all ratios given are w/w). These ratios can be for CBD:THC or THC:CBD or applicable to the cannabinoids selected.

“Cannabinoid,” as used herein, is further meant to include compounds which interact with the cannabinoid receptor and various cannabinoid mimetics, such as certain tetrahydropyran analogs (e.g., Δ9-tetrahydrocannabinol, Δ8-tetrahydrocannabinol, 6,6,9-trimethyl-3-pentyl-6H-dibenzo[b,d]pyran-1-ol, 3-(1,1-dimethylheptyl)-6,6a,7,8, 10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylhept-yl, (+)-(3 S,4S)-7-hydroxy-Δ6-tetrahydrocannabinol-1,1-dimethylh-eptyl, 11-hydroxy-Δ9-tetrahydrocannabinol, and Δ8-tetrahydrocannabinol-11-oic acid)); certain piperidine analogs (e.g., (−)-(6S,6aR,9R, 10aR)-5,6,6a,7,8,9, 10,10a-octahydro-6-methyl-1-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenylmethanone), certain open pyran ring analogs (e.g., 2-[3-methyl-6-(1-methylethenyl)-2-cyclohexen-1-yl]-5-pentyl-1,3-benzenedi-ol and 4-(1,1-dimethylheptyl)-2,3′-dihydroxy-6′alpha-(3-hydroxypropyl)-1′,-2′,3′,4′,5′,6′-hexahydrobiphen-yl), as well as their pharmaceutically acceptable salts, solvates, metabolites (e.g., cutaneous metabolites), and metabolic precursors.

“Δ9-THC,” as used herein, is meant to refer to Δ9-tetrahydrocannabinol as well as to its pharmaceutically acceptable salts, solvates, metabolites (e.g., cutaneous metabolites), and metabolic precursors. Δ9-tetrahydrocannabinol is marketed under the generic name “dronabinol.”

“Cannabinol,” (CBN) as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6H-dib-enzo[b,d]pyran-1-ol.

“Cannabidiol,” (CBD) as used herein, is meant to refer to 2-[3-methyl-6-(1-methylethenyl)-2-cyclohexen-1-yl]-5-pentyl-1,3-benzenedi-ol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenedi-ol.

“Nabilone,” as used herein, is meant to refer to 3-(1,1-dimethylheptyl)-6,6a,7,8,10,10a-hexahydro-1-hydroxy-6,6-dimethyl-9-H-dibenzo[b,d]pyran-9-one as well as to pharmaceutically acceptable salts, solvates, metabolites (e.g., cutaneous metabolites), and metabolic precursors of 3-(1,1-dimethylheptyl)-6,6a,7,8,10,10a-hexahydro-1-hydroxy-6,6-dimethyl-9H-dibenzo[b,d]pyran-9-one.

“Levonantradol,” as used herein, is meant to refer to (−)-(6S,6aR,9R,10aR)-5,6,6a,7,8,9,10,10a-octahydro-6-methyl-3-[(R)-1-meth-yl-4-phenylbutoxy]-1,9-phenan-thridinediol 1-acetate, as well as to pharmaceutically acceptable salts, solvates, metabolites (e.g., cutaneous metabolites), and metabolic precursors of (−)-(6S,6aR,9R, OaR)-5,6,6a,7,8,9,10,10a-octahydro-6-methyl-3-[(R)-1-methyl-4-phenylbuto-xy]-1,9-phenanthridinediol 1-acetate. (−)-(6S,6aR,9R,10aR)-5,6,6a,7,8,9,10,10a-octahydro-6-methyl-3-[(R)-1-methyl-4-phenylbuto-xy]-described in U.S. Pat. Nos. 4,206,225, 4,232,018, and 4,260,764, which are hereby incorporated by reference; in U.S. Pat. No. 4,235,913 which is hereby incorporated by reference; in U.S. Pat. No. 4,243,674 which is hereby incorporated by reference; and in U.S. Pat. Nos. 4,263,438, 4,270,005, and 4,283,569, which are hereby incorporated by reference.

“(−)-HU-210,” as used herein, is meant to refer to (−)-(3S,4S)-7-hydroxy-Δ6-tetrahydrocannabinol-1,1-dimethylhept-yl as well as to pharmaceutically acceptable salts, solvates, metabolites (e.g., cutaneous metabolites), and metabolic precursors of (−)-(3S,4S)-7-hydroxy-Δ6-tetrahydrocannabinol-1,1-dimethylhept-yl. (−)-(3S,4S)-7-hydroxy-Δ6-tetrahydro-cannabinol-1,1-dimethylheptyl is particularly useful in pain control, and its preparation is described in U.S. Pat. Nos. 4,876,276 and 5,521,215, which are hereby incorporated by reference.

“(+)-HU-210,” as used herein, is meant to refer to (+)-(35,45)-7-hydroxy-Δ6-tetrahydrocannabinol-1,1-dimethylhept-yl as well as to pharmaceutically acceptable salts, solvates, metabolites (e.g., cutaneous metabolites), and metabolic precursors of (+)-(3S,4S)-7-hydroxy-Δ6-tetrahydrocannabinol-1,1-dimethylhept-yl. (+)-(3 S,4S)-7-hydroxy-Δ9-tetra-hydrocannabinol-1,1-dimethylh-eptyl is sometimes referred to as HU-211 and/or dexanabinol; it is an antagonist of the N-methyl-D-aspartate receptor; and is described in U.S. Pat. Nos. 4,876,276 and 5,521,215, which are hereby incorporated by reference.

“11-hydroxy-Δ⁹-THC,” as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahyd-rocannabinol is a more hydrophilic, psychoactive metabolite of Δ⁹-tetrahydrocannabinol, and its laboratory synthesis is described in Siegel et al., J. Org. Chem., 54:5428 (1989), which is hereby incorporated by reference.

“Δ⁸-THC-11-oic acid,” as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabino-1-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa). Δ8-tetrahydrocannabinol-11-oic acid can also be produced synthetically as set forth in U.S. Pat. No. 6,162,829, which is hereby incorporated by reference. Δ⁸-tetrahydrocannabin-ol-11-oic acid is more hydrophilic than 6a,7,10,10a-tetrahydro-6,6,9-trimethyl-3-pentyl-6H-diben-zo[b,d]pyran-1-ol, and it has analgesic activity.

“CP 55,940,” as used herein, refers to 4-(1,1-dimethylheptyl)-2,3′-dihydroxy-6′alpha-(3-hydroxypropyl)-1′,2′,3′,4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′alpha-(3-hydroxypropyl)-1′,2′,3′,4′,5′,6′-hexahydro-biphenyl is sometimes referred to as (−)-cis-3-[2-Hydroxy-4-(1,1-dimethylheptyl)phenyl]-trans-4-(3-hydroxyprop-yl)cyclohexanol, and has been described in U.S. Pat. Nos. 4,371,720 and 4,663,474 which are hereby incorporated by reference.

“R(+)-WIN 55,212-2,” as used herein, refers to (R)-(+)-[2,3-dihydro-5-methyl-3-(4-morpholinylmethyl)-pyrrolo[1,2,3-de]-1-,4-benzoxazin-6-yl]-1-naphthalenyl-methanone, as well as to its pharmaceutically acceptable salts, solvates, metabolites (e.g., cutaneous metabolites), and metabolic precursors. (R)-(+)-[2,3-dihydro-5-methyl-3-(-4-morpholinylmethyl)-pyrrolo[1,2,3-de]-1,4-benzoxazin-6-yl]-1-naphthalenyl-1-methanone (in its mesylate form).

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 which is hereby incorporated by reference.

An “effective amount” as used herein, means an amount which provides a therapeutic or prophylactic benefit.

The term “therapeutically effective amount” or “therapeutically and/or prophylactically effective amount” as used herein refers to an amount of a cannabinoid that is sufficient to elicit the required or desired therapeutic and/or prophylactic response. Typically the “therapeutically effective amount” or “therapeutically and/or prophylactically effective amount” of cannabinoid is sufficient to alleviate one or more symptoms associated with pain and/or one or more symptoms associated with a Cannabis- or cannabinoid-treatable condition. For example, while pain is provided herein as a specific exemplary treatable condition/symptom, the compositions described herein may find use in treating any condition in which Cannabis or a Cannabis extract is useful. For example, cannabinoids, natural or synthetic, are typically agonists at cannabinoid receptors and many diseases or conditions or symptoms of such diseases or conditions can be alleviated at least in part by the administration of cannabinoid receptor agonists. Other compounds within Cannabis, in the form of an extract or purified compound or mixture of compounds, may also find use in the compositions described herein.

A therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age.

The term “pharmaceutically acceptable” means that the compound or combination of compounds is compatible with the remaining ingredients of the formulation for pharmaceutical use, and that it is generally safe for administering to humans according to established governmental standards.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The presently developed novel cannabinoid delivery system provides for biphasix multilayered lipid vesicles to be made that have several internal compartments loaded with a cannabinoid. The combination of aqueous compartments, bilayer compartments, micellar compartments and oily compartments provides for the cannabinoid to be able to be formulated within the desired compartments for more controlled and yet greater formulation versatility. Thus the cannabinoid delivery system will be more stable and provide for an improved therapeutic effect. The system can release the cannabinoid quickly or for a longer duration as it is uptaken into the skin or mucosa. Thus rapid cutaneous times are achievable as are a more depot formation within the skin with a slow release of the cannabinoid for pain alleviation and management.

Diseases and conditions are that of pain and conditions that include pain as a symptom. Such diseases and conditions include but are not limited to the following: pain (including but not limited to acute pain; chronic pain; neuropathic pain and cancer pain), immunomodulation (such as increasing a positive immune response or decreasing a negative immune response, or inducing tolerance to an immunogenic agent), neurodegenerative disease (including but not limited to Alzheimer's disease; Parkinson's disease; amyotrophic lateral sclerosis; Huntington's disease; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; epilepsy or any other related neurological or psychiatric neurodegenerative disease), ischemic disease (including but not limited to stroke; cardiac ischemia; coronary artery disease; thromboembolism; myocardial infarction or any other ischemic related disease), brain injury or damage (including but not limited to traumatic brain injury including: diffuse axonal injury; concussion; contusion; whiplash or any other traumatic head or brain injury), acquired brain injury (including but not limited to stroke; anoxic brain injury; hypoxic brain injury or any other acquired brain injury), age related inflammatory or autoimmune disease, cachexia (including related conditions such as AIDS wasting disease, weight loss associated with cancer, chronic obstructive pulmonary disease or infectious diseases such as tuberculosis), nausea and vomiting, glaucoma, movement disorders, rheumatoid arthritis, asthma, allergy, psoriasis, Crohn's disease, systemic lupus erythematosus, diabetes, cancer, osteoporosis, renal ischemia and nephritis.

In particular aspects, the cannabinoid compositions and formulations described herein may find use, for example, as an analgesic for treatment of pain associated where the composition/formulation can be locally applied. Thus the compositions or formulations containing such are suitable for topical and transdermal administration to an area of pain on skin or mucosa. The skin may healthy and not compromised in which the compositions of the invention can be administered repeatedly vigorously massaged into the skin to raise the skin temperature such that the composition may penetrate pores in the epidermis of the skin for transdermal administration. The composition can also be applied to compromised skin where the compromised skin comprises damage to the stratum corneum. The skin can be physically compromised by cuts, scrapes, wounds, bites, incisions, blisters and/or punctures. Topical administration of the composition of the invention as well as formulations incorporating the composition of the invention helps to alleviate pain during healing of compromised skin. Compromised skin may also be due to a dermatological condition such as inflammation and is selected from acne, hives, psoriasis, heat burns, sunburn, chemical burns, dermatitis, keratosis, rosacea, carbuncle, eczema, cellulitis, measles, lupus or impetigo. Topical administration of the composition or formulations of the invention helps to alleviate pain, irritation and inflammation of the dermatological condition.

Topical administration can also be applied to mucosa of the mouth, nose, vagina or rectum to relieve pain and irritation.

In any aspect, the Cannabis compositions of the invention or formulations containing such may be administered repeatedly to said skin. This can be repeated several times a day, each days continually as desired.

The cannabinoid amount, in terms of weight percent, in the biphasix multilayered lipid vesicle composition is generally between about 0.01% and about 15% of the composition, between about 0.05% and about 4%, between about 0.1% and about 3.5%, between about 0.2% and about 3%, between about 0.4% and about 2%, or between about 0.6% and about 1.5%. In one embodiment, the amount is between about 0.8% and 1.2%, or about 1%. Alternatively, the cannabinoid amount can be about 0.01%, about 0.05%, about 0.1%, about 0.2%, about 0.4%, about 0.6%, about 0.8%, about 0.9%, about 1%, about 1.1%, about 1.2%, about 1.4%, about 1.6%, about 1.8%, about 2%, about 3%, about 4%, about 5%, about 6%, about 8%, about 10%, about 15%, or about 20%. The remaining ingredients are adjusted to maintain the desired weight percent of the desired cannabinoid.

In aspects of the invention the cannabinoid can be provided as a powderized Cannabis oil which comprises or consists essentially of Cannabis oil and maltodextrin as taught in U.S. Pat. No. 9,629,886 (the disclosure of which is incorporated herein in its entirety). In aspects the powderized Cannabis oil is CBD/maltodextrin, decarboxylated CBD/maltodextrin. The amount of CBD contained in the powderized Cannabis can be readily determined and made to vary, for example up to 200 milligrams CBD in a tablet or capsule.

In some embodiments individual doses of the compositions of the present invention contain from about 0.1 to about 100 milligrams (mg) of cannabinoid, from about 0.5 to about 50 mg, from about 1 to about 40 mg, from about 2 to about 20 mg, from about 5 mg to about 15 mg, or about 0.1 mg., 0.5 mg, 1 mg, 2 mg, 3 mg, 4 mg, 5 mg, 6 mg, 8 mg, 10 mg, 12 mg, 14 mg, 16 mg, 18 mg, 20 mg, 30 mg, 40 mg, 50 mg, 60 mg, 80 mg or more per dose.

For transdermal delivery of the composition of the invention involves contacting the composition comprising one or more cannabinoids with the subject's skin under conditions effective for at least one of the provided cannabinoids to penetrate the skin and enter the bloodstream. The compositions of the present invention allow for significant transdermal delivery across compromised skin. A number of methods known in the art can be used to assess delivery across the skin. In one method, delivery may be assessed by measurement of the remaining cannabinoid in the composition after use. After the composition was present on the skin of a patient for at least 12 hours, for example, at least 0.1% of the cannabinoid can be delivered across the skin, at least 0.5% of the cannabinoid can be delivered across the skin, at least 1% of the cannabinoid can be delivered across the skin, at least 2% of the cannabinoid can be delivered across the skin, at least 3% of the cannabinoid can be delivered across the skin, at least 4% of the cannabinoid can be delivered across the skin, at least 5% of the cannabinoid can be delivered across the skin, at least 6% of the cannabinoid can be delivered across the skin, at least 7% of the cannabinoid can be delivered across the skin, at least 8% of the cannabinoid can be delivered across the skin, at least 9% of the cannabinoid can be delivered across the skin, at least 10% of the cannabinoid can be delivered across the skin, at least 11% of the cannabinoid can be delivered across the skin, at least 12% of the cannabinoid can be delivered across the skin, at least 14% of the cannabinoid can be delivered across the skin, at least 16% of the cannabinoid can be delivered across the skin, at least 18% of the cannabinoid can be delivered across the skin, at least 20% of the cannabinoid can be delivered across the skin, at least 25% of the cannabinoid can be delivered across the skin, at least 30% of the cannabinoid can be delivered across the skin, at least 35% of the cannabinoid can be delivered across the skin, at least 40% of the cannabinoid can be delivered across the skin, at least 45% of the cannabinoid can be delivered across the skin, at least 50% of the cannabinoid can be delivered across the skin, at least 55% of the cannabinoid can be delivered across the skin, at least 60% of the cannabinoid can be delivered across the skin, at least 65% of the cannabinoid can be delivered across the skin, at least 70% of the cannabinoid can be delivered across the skin, at least 75% of the cannabinoid can be delivered across the skin, at least 80% of the cannabinoid can be delivered across the skin, at least 85% of the cannabinoid can be delivered across the skin, at least 90% of the cannabinoid, at least 90% of the cannabinoid can be delivered across the skin, and at least 95% of the cannabinoid can be delivered across the skin.

The cannabinoid or mixture thereof is provided in the first and/or second phase of the biphasix vesicle composition. Each phase having additional compartments. Optionally, opioids can be combined with the cannabinoids in the first and/or second phase of the biphasix vesicle composition or as an adjunct to the compositions of the invention. Opioids are often used for acute pain, such as short-term pain after surgery. Some examples of opioids include but are not limited to: morphine, fentanyl, oxycodone and codeine. Other components may also be included in the compositions described herein, as will be understood.

In aspects, the compositions described herein may act additively or synergistically with other conventional anti-pain treatments, whether administered concurrently or consecutively in any order and/or whether administered topically or by any other known method.

In aspects, the compositions described herein may be formulated with a non-steroidal anti-inflammatory drug (NSAID) such as salicylic acid and oxyacetic acid. Other examples of NSAID that can be used include salicylates, propionic acid derivatives, acetic acid derivatives, enolic acid derivatives, fenamic acid derivatives, coxibs, sulphonanilides, etc. In some preferred embodiments, the NSAID can be acetylsalicylic acid or Bendazac. This may be particularly useful in the treatment of eye pain when the composition is formulated as an eye drop.

By format, it is meant that the cannabinoid biphasix multilayered lipid vesicle compositions of the invention can be provided formulated as an ointment, cream, suspension, liquid, lotion, paste, gel, spray, foam, oil, semi-solid (i.e. suppository), bar (soap bar), shampoo and combinations thereof. Any of these formats as suitable can be incorporated, for example, into a patch for transdermal administration or into a suppository for transmucosal administration. One of skill in the art would understand how to prepare a formulation for application. For example, a cream formulation comprises at least two ingredients selected from the group consisting of water, ceteraryl octanoate, glycerin, shea butter, sweet almond oil, palm oil, jojoba oil, aloe barbaensis, maris sal, potassium sorbate, sclerotium gum, xanthum gum, tocopheryl acetate, camellia sinensis leaf extract, corral powder and any combination thereof.

The invention relates to a lipid-bilayer or liposome or lipid vesicle composition for use in delivering a cannabinoid by topical application meaning the provision of a local effect, where the composition is applied directly where its action is desired. The term topical may be defined as application to a localized area of the body or to the surface of a body part, without necessarily involving a targeted effect of the substance, resulting in a systemic effect. Examples of topical administration/use includes, for example, transdermal, transmucosal delivery (e.g., by intravaginal administration, rectal, or intranasal) and ocular. In aspects, there are also localized benefits from topical administration. For example, topically administered cannabinoids may find use in alleviating pain and other conditions originating near the surface of the skin. Transdermal includes application to any skin portion of the body.

In one embodiment, compositions described herein are suitable for transdermal administration. Transdermally administrable compositions are adapted for administration in and/or around the abdomen, back, chest, legs, arms, scalp or other suitable skin surface and may include formulations in which the cannabinoid biphasix multilayered lipid vesicle composition is administered in patches, ointments, creams, suspensions, liquids, lotions, pastes, gels, sprays, foams, soaps, shampoos or oils.

In an further embodiments, the composition of the invention is formulated to be applied to the eye as a drop, in this type of embodiment the composition can be a liquid, liquid suspension or gel that is not overly viscous. Furthermore, as an eye drop the composition of the invention may additionally comprise a lubricant (e.g., glycerin, polysorbate, hypromellose, hydroxyethyl cellulose, carboxymethylcellulose, etc.), a redness reliever (e.g., naphazoline hydrochloride, tetrahydrozoline, etc.), an astringent (e.g., zinc sulfate, etc.) or various inactive ingredients (e.g., borate buffer, silver sulphate preservative, benzalkonium chloride, boric acid, chlorobutanol, edentate disodium, menthol, sodium borate, calcium chloride, magnesium chloride, potassium chloride, sodium chloride, sodium lactate, a pH adjuster (e.g., hydrochloric acid, sodium hydroxide, etc.), a buffer, etc.). Antibiotics such as chloramphenicol, fusidic acid, fluoroquinolones, aminoglycoside and polymycin B sulfate may also be incorporated into the composition of the invention or used concurrently therewith.

After transdermal application of the composition, essentially no limitations exist as to the length of time that the composition can remain in contact with the user's skin. Since the amount of Cannabis in the composition will decrease as it is absorbed into the user's skin, the composition can be removed when the amount of cannabinoid remaining in the composition decreases to an amount that is no longer effective to the user. It is to be understood that the amount of cannabinoid initially carried in the composition will affect the length of time the composition will be effective once the composition is applied to the user's skin. For example, in an aspect of the invention, the composition contains a cannabinoid in the amount of about 10 milligrams. In such an aspect, the composition may be removed after approximately 12 hours, and after that time replaced with a new dose of the composition for continued absorption of cannabinoid into the user's skin to provide therapeutic levels of the cannabinoid to the user. However, the composition may optionally be left on longer than, or removed sooner than, the length of time that is necessary or recommended for complete diffusion of the cannabinoid into the user's skin. As mentioned above, the composition of the present invention placed on the skin is capable of delivering Cannabis through the stratum corneum layer of the epidermis and through the dermis into the microvasculature.

“Alleviate” as used herein, is meant to include complete elimination as well as any clinically or quantitatively measurable reduction in the subject's symptoms and/or discomfort.

By pain as used herein is meant both acute and chronic. For example acute pain usually comes on suddenly and is caused by something specific. It is sharp in quality. Acute pain usually does not last longer than six months. It goes away when there is no longer an underlying cause for the pain. Causes of acute pain include: surgery, broken bones, dental work, burns, cuts, strains, sprains, pain due to intercourse and the like. Chronic pain is pain that is ongoing and usually lasts longer than six months. This type of pain can continue even after the injury or illness that caused it has healed or gone away. Pain signals remain active in the nervous system for weeks, months, or years. Some people suffer chronic pain even when there is no past injury or apparent body damage. Chronic pain is linked to conditions including but not limited to: headache, arthritis, cancer, nerve pain, back pain, fibromyalgia, bursitis, carpal tunnel syndrome, gout, and other muscular and joint aches and pains.

Neuropathic pain is generated by pathology in the peripheral or central nervous system. A large number of disorders can give rise to neuropathic pain. This may range from nerves being cut (trauma or surgery) or damaged by viruses, ischemic and metabolic injury or complex genetic disorders to name a few. Neuropathic pain may arise from local damage to neural tissues as well as tissues remote to initial trauma and may also arise as a result of chronic inflammatory disease. Pharmacological management is one of the most used pain treatment options but results are poor with many patients obtaining inadequate relief with currently available agents. There is therefore a need for new agents for treatment of neuropathic pain. Neuropathic pain may affect any part of the body including the eye for which there are no adequate treatments at present.

The composition of the invention has use to help prevent or relieve pain associated with the eyes associated with at least one of the following eye disorders: (a) cataracts; (b) diabetic retinopathy; (c) glaucoma; (d) macular degeneration; (e) dry eye syndrome (e.g., irritated eyes, sandy or gritty sensation, red eyes, burning sensation, poor visual acuity, poor tear quality, decreased tear break up time, poor schrimer test performance, increased eye sensitivity to wind and heat, etc.); (f) proptosis (e.g., dryness, eye pain, eye redness, etc.); (g) keratoconus; (h) pterygium/pinguecula (e.g., distorted vision, blurred vision, decreased visual acuity, inflammation, irregular astigmatism, etc.); (i) ocular allergy (e.g., eye irritation, blurred vision, decreased visual acuity, etc.; or any other eye disorders and signs or symptoms.

Pain associated with uveitis, an intraocular inflammation within the eye from the uvea (iris, ciliary body and choroid) to the sclera, retina and optic nerve is also encompassed within the scope of the present invention. It involves either infectious or non-infectious conditions, which can be localized within the eye or associated with systemic inflammatory and autoimmune diseases, including reactive arthritis and multiple sclerosis. The most common form of uveitis, anterior uveitis, with inflammation of the iris and ciliary body, is additionally associated with considerable pain and photophobia (Jabs, Nussenblatt et al. 2005; Lee and Dick 2012). Untreated uveitis can lead to permanent loss of vision. Severe uveitis is treated aggressively to mitigate the damage caused by inflammation.

Anterior uveitis (iritis) is associated with inflammation of iris and anterior tissues and this leads to pain and light sensitivity with pupillary changes in response to light. Anterior uveitis pain is typically resolved when the inflammation is treated so is not classed as neuropathic pain. Generally uveitis represents hyperactivation of the body's immune system; a form of local sepsis. Inflammatory conditions are represented by activation, recruitment, and migration of immune cells, release of proinflammatory cytokines, swelling, oedema and/or tissue damage. In posterior uveitis, this can also include gliosis, and activation of resident immune cells (microglia). In some retinal inflammatory diseases, cell proliferation with subsequent fibrosis and retinal detachment is present (i.e. proliferative vitreoretinopathy).

Corneal neuropathic hyperalgesia involves a dysfunctional corneal pain system and is associated with significant discomfort and persistent heightened sensitivity of the cornea (peripheral sensitization) in the absence of overt trauma or noxious stimuli (reviewed in Belmonte et al., 2004; Rosenthal & Borsook, 2012; Rosenthal et al., 2009). Ongoing excitation of corneal nerves, following corneal damage or irritation, results in the release of neuropeptides and inflammatory mediators that augment the inflammatory reaction (neurogenic inflammation) leading to hyperalgesia. Corneal hypersensitivity, neuroinflammation, pain and photophobia are reported in patients following refractive surgery and chemical/toxic exposure, including repetitive use of benzalkonium chloride-preserved eye drops. Corneal neuropathic pain is also a central pathogenic feature of eye disorders that are collectively referred to as dry eye, and include non-infectious immunological causes such as Sjogren syndrome and systemic lupus as well as infections with Herpes Zoster (reviewed in Rosenthal & Borsook, 2012; Yawn et al., 2013).

In aspects, the cannabinoid biphasix vesicle composition described herein comprises a) a first phase comprising an oil-in-water emulsion which itself comprises oil in water, wherein a sufficient amount of oil is employed to form a composition suitable for topical application, and wherein the water comprises a cannabinoid, an optional antioxidant and an optional anti-aggregant; and (b) a second phase comprising multilamellar lipid vesicles suspended in said first phase wherein said vesicles contain entrapped therein a composition comprising an oil-in-water emulsion wherein the water phase comprises cannabinoid, an optional antioxidant and an optional anti-aggregant, wherein the composition comprises a therapeutically effective amount of said cannabinoid, and wherein said anti-aggregant preserves the cannabinoid so as to enhance the shelf-life of the composition. The composition surprisingly provides for the loading of at least one cannabinoid in a manner that is stable and is released when topically applied in a manner to help alleviate pain and pain associated with a variety of conditions as described herein. In aspects the composition comprises CBD, whether in one of the phases of the vesicle or separately in different phases of the vesicle or all phases. One of skill in the art would appreciate that the vesicles can comprise more than two phases and each can be loaded with a desired cannabinoid such as CBD and another cannabinoid in desired ratios.

The composition described herein is, in aspects, safe and effective and may find particular advantages for use. For example, the compositions described herein may bypass many of the euphoric activities of THC and enable more effective transdermal delivery of cannabinoids into the dermis.

It can in aspects be used in a variety of formats and also applied to a patch (to form a Cannabis transdermal delivery structure) that is constructed to have a backing layer selected from the group consisting of a patch, strip, bandage or covering, for example, the backing layer comprising the composition of the invention and optional other skin permeation enhancer(s) or other components. One of skill in the art would recognize that the composition described herein can be incorporated into a variety of patch formats such as for example but not limited to those disclosed in U.S. Pat. Nos. 6,113,940, 6,328,992 and 9,375,417 each of which are incorporated herein by reference in their entirety.

The cannabinoid biphasix multilayered lipid vesicle composition of the invention can be provided as a kit with instructions for use depending on the format of the composition.

The cannabinoid biphasix multilayered lipid vesicle composition of the invention may be effective for the treatment of all types of pain whether acute or chronic or as a result of injury, surgery, or disease state. The cannabinoid biphasix multilayered lipid vesicle composition of the invention can be applied topically to any part of the body inclusive of orifices and to the eye as drops for example.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

### EXAMPLES

The following examples are provided for illustrative purposes only and are not intended to limit the scope of the invention.

### Example One: Method of Making Biphasix Multilamellar Liposome Cannabinoid Composition

A multilamellar lipid vesicle is made as follows. An oil and a consistency enhancer are admixed. Separately, water and a surfactant are admixed. A water-soluble antimicrobial agent, for example methyl paraben or propylparaben, a buffering agent, such as phosphates, and a chelating agent, such as EDTA, can also be dissolved in the water and heated to about 70° C., and then admixed and homogenized with the oil and consistency enhancer. This results in formation of an emulsion with water as the continuous phase and the oil and consistency enhancer as the dispersed phase. It is desirable that the oil droplets shall be less than about 1 μm, especially less than about 0.5 μm, in diameter and if necessary the emulsion can be subjected to additional shear or to sonification to reduce the size of the droplets.

Separately prepared is an anhydrous proliposome gel by admixing phospholipid, glycolipid and/or ceramide and a pharmaceutically acceptable hydrophilic solvent, e.g., propylene glycol, and heating to form a melt. In the melt there may also be incorporated a material to enhance the strength of the lipid bilayers, for example cholesterol, a material to enhance penetration, for example monolauroyllysine and a material to impart a charge to the lipid bilayers, for example stearic acid. A small amount of an antioxidant, for example ascorbyl palmitate, butylated hydroxytoluene or butylated hydroxyanisole can be incorporated in the melt. Further, TPGS or Vitamin E TPGS can also be incorporated for stabilizing the melt. The aqueous emulsion is added to the melt and the various components are subjected to agitation which results in formation of the desired multilamellar lipid vesicles having in the central core compartment an aqueous emulsion containing the oil and consistency enhancer as the dispersed phase. The cannabinoid is incorporated into the emulsion.

### Formation of an Anhydrous Plastic Proliposome Gel

A liposome-forming component and other necessary excipients are melted with a pharmaceutically acceptable hydrophilic solvent, such as propylene glycol.

The expression “liposome-forming component” designates the substance or substances used as major component of the lipid bilayers. Typical liposome-forming components include glycolipids, lecithins, phospholipids, ceramides or mixtures thereof which are used as a primary ingredient in the formation of the lipid bilayer. However, other natural and synthetic compounds having the required amphipatic character can be incorporated with the phospholipid, glycolipid or ceramide, replacing some of these expensive materials, provided that the essential character of the lipid bilayers is not adversely affected. The choice of the appropriate materials is within the knowledge of the person skilled in the art. Examples include phosphatidylethanolamine, lysolecithin, lysophosphatidylethanolamine, phosphatidylserine, phosphatidylinositol, sphingomyelin, cardiolipin, phosphatidic acid and the cerebrosides, ether lipids and phytanols.

The liposomal formulations of the present invention comprise saturated and/or unsaturated phospholipids, more preferably phosphatidylcholine, lysophosphatidylcholine, phosphatidylserine, phosphatidylethanolamine, glycolipids and ceramides. The phospholipids may be used in combination with a penetration enhancing agent such as monolauroyllysine, dipalmitoyllysine or methyl salicylate to achieve predominantly transdermal delivery potential.

A “fatty substance” can be used to enhance the strength of the lipid bilayers. Examples of useful fatty substances include steroids such as cholesterol, coprostanol, cholestanol and cholestane and long chain fatty acids (C₁₆ to C₂₂), especially saturated ones such as stearic acid. In addition to enhancing strength of the lipid bilayer, acids impart a negative charge. Saturated or unsaturated acids can be used. Other fatty substances that can be used include C₁₆ to C₂₂ fatty amines, fatty acylated proteins, fatty acylated peptides, fatty acylated PEG and derivatives. These fatty substances are incorporated with the abovementioned liposome-forming components and improve physical stability and appearance of the product.

The hydrophilic solvent is used as a plasticizer of the liposome-forming component and an aid to prepare a uniform melt. Examples of hydrophilic solvents include but are not restricted to propylene glycol, glycerol, polyethylene glycol having a molecular weight ranging between 300 and 8000, ethanol, and mixtures thereof. The resulting melt can be described as being an anhydrous plastic proliposome gel. This anhydrous plastic proliposome gel contains all the lipid phase ingredients and can be prepared and stored in advance in large quantities. It is a semisolid material with a homogenous consistency.

### Formation of Multilamellar Lipid Vesicles (MLV)

Hydrophilic ingredients such as penetration enhancers, preservatives and the like, are prepared separately as an aqueous solution, which forms the continuous phase of an emulsion. This is added to the lipid phase melt, previously heated to the appropriate melting temperature that can range from 40° C. to 80° C., and vigorously mixed by any given technique which allows the achievement of the desired product size. Examples of mixing techniques include vortexing or propeller mixing. At this stage, it is also possible to incorporate (dissolve) the cannabinoids that will be entrapped within the lipid bilayers.

This procedure is suitable for the preparation of various amounts of topical liposomal product. If vortex mixing is used as the agitation, up to about 20 g of the product can be prepared. If a laboratory scale propeller mixer is used, up to about 2 kg to 10 kg of the product can be made. This formulation procedure can also be adapted for large scale manufacturing. Hence, the propeller mixing technique can be directly scaled up by geometrically increasing the size of the vessel and the diameter of the propeller mixer. However, as the vessel size increases, the preferred set up would be a combination mixer, i.e. a high intensity mixer with propeller mixer and a scraped surface agitator. The aqueous phase can either be pumped from tank A to tank B containing the anhydrous plastic proliposome gel or the aqueous phase can be mixed with the emulsion prior to adding to Tank B at the required temperature and mixed. This procedure is suitable for the production of any topical liposomal product on a large scale.

Liposomal compositions can be prepared with the multilamellar lipid vesicles of the present invention by using appropriate pharmaceutical additives. For example, it might be required to add viscosity increasing agents to the final liposome preparation. The addition of other pharmaceutically acceptable compounds in addition to the cannabinoids is within the purview of the person skilled in the art.

### Characteristics of the Final Multi-Lamellar Lipid Vesicle Product

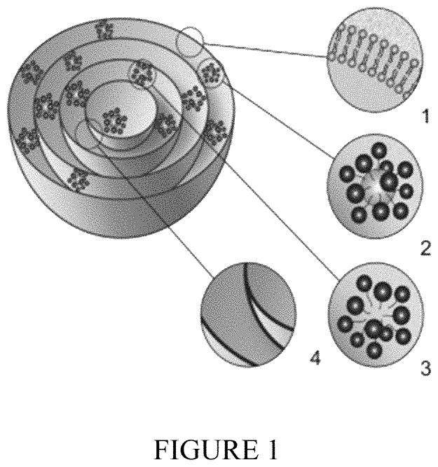

A schematic representation of the structure, assembly and properties of the biphasix multilayered lipid vesicle is shown in FIG. 1. Lipophillic active cannabinoid is incorporated into the oil phase of the submicron emulsion. Vesicles of concentric phospholipid bilayers (1); cationic submicron emulsion droplets (2); cationic surfactant micelles (3) and water phase (4) are shown. Cannabinoid can be provided within any one or more or all of these compartments.

It will be appreciated that optional stabilizers, optional anti-aggregant and an optional water soluble antioxidant as well as any other desired components will be present in the water of the aqueous emulsion in the central core compartment and in the peripheral compartments. Other inactive ingredients that are lipophilic, such as consistency enhancers or uptake enhancers, can be present in the dispersed phase of the emulsion in the central compartment and in the peripheral compartments. They can also be present in the interior of the lipid bilayers.

In various aspects of the invention, an anti-aggregant, such as arginine, may be present in the intra-vesicular and extra-vesicular spaces of the multilamellar vesicles.

The term “stability” refers to the physical, chemical, and/or conformational stability of formulations of cannabinoid of the invention (including maintenance of biological potency).

A “stable” or “stabilized” composition is one wherein the degree of degradation, modification, aggregation, loss of biological activity and the like, of the cannabinoid therein is acceptably controlled, and does not increase unacceptably with time. Typically, the composition retains at least or about 60%, more typically at least at or about 70%, most typically at least at or about 80% of the labeled cannabinoid activity over a period of 24 months. The stabilized cannabinoid compositions of the invention preferably have a shelf-life of at least about 18 months, more preferably at least 20 months, still more preferably at least about 22 months and most preferably at least about 24 months when stored under refrigerated conditions (2° C.-8° C.).

In various aspects, one or more antioxidants may be included in the formulations according to the invention, and in certain aspects a combination of two or more antioxidants is employed. In embodiments, the antioxidant employed is L-methionine, although it is also contemplated that D-methionine can be used, or alternatively a racemic mixture of both. Thus, any stereoisomer (i.e., L, D or DL isomer) of methionine may be used in the compositions of the invention. Analogues of methionine may also be used, the term “methionine analogue” referring to a derivative of the naturally occurring methionine, for instance, methionine derivatives with alpha and/or beta-amino substituted groups. In exemplary embodiments, the amount of methionine used in the composition may range from about 0.01 to about 5 weight percent based on the total weight of the composition. More preferably, the amount of methionine ranges from about 0.01 to about 0.5 weight percent based on the total weight of the composition.

The composition may further comprise at least one additional antioxidant to further stabilize cannabinoid in the biphasix lipid vesicles. Additional antioxidants include, but are not limited to, ascorbic acid and its salts, ascorbyl palmitate, ascorbyl stearate, N-acetylcysteine, benzyl isothiocyanate, caffeic acid, sodium metabisulfate, benzyl alcohol and tocopherols, including alpha-tocopherol and its salts.

Further, the term “anti-aggregant” as used herein refers to any biocompatible compound that inhibits and/or reduces the aggregation of the active, e.g., formation of aggregates of active. The process of aggregation can be influenced by a variety of factors, such as but not limited to physicochemical stresses, including heat, pressure, pH, agitation, shear forces, freeze-thawing, dehydration, heavy metals, oxygen, phenolic compounds, silicon oil, denaturants and the like. To the degree that any is required with respect to cannabinoid aggregation in the MVL, such can be incorporated.

The term “guanidine” as used herein includes guanidine and derivatives thereof (e.g., in which the hydrogen atom attached to the amidino nitrogen is replaced by substituted or unsubstituted carboxyl groups, substituted or unsubstituted amino groups, substituted or unsubstituted alkyl groups, substituted or unsubstituted heteroalkyl groups, substituted or unsubstituted aryl groups, and substituted or unsubstituted heteroaryl groups). In preferred embodiments, the anti-aggregants include compounds that contain a guanidine group, for example, guanidinoacetic acid, substituted or unsubstituted guanidinobenzoic acid, guanidine carbaniedine, guanidine acetate, guanidine amine, guanidine carbonate, guanidine nitrate, guanidine hydrochloride, arginine, arginine analogues and the like. Arginine that has been derivatized at the carboxy or alpha-amino groups is also contemplated. In a preferred embodiment, L-arginine hydrochloride is used as an anti-aggregant.

A pharmaceutically acceptable salt of arginine may impart enhanced shelf-life to the composition by reducing the formation of aggregates. The arginine employed is preferably a pharmaceutically acceptable salt of L-arginine although it is contemplated that D-arginine can also be used, as can a racemic mixture of both. Suitable pharmaceutical salts include, by way of example only, well known organic and inorganic salts such as hydrochloride salts, hydrobromide salts, C₁ to C₆ carboxylic acid salts such as acetate, proprionate, succinate, oxalate, benzoate salts. The amount of pharmaceutically acceptable salt of arginine used in the composition ranges from about 0.01 to about 5 weight percent based on the total weight of the composition.

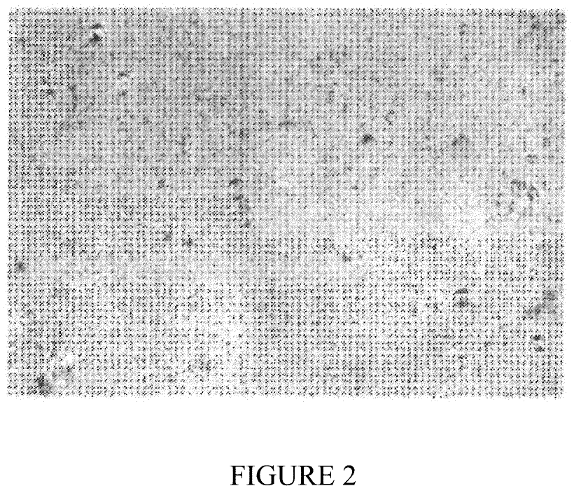

FIG. 2 is an image, magnified 440×, of cannabinoid vesicles made for use as a topical lotion and exhibits the consistency of a lotion or semi-solid cream. The multi-lamellar structures have a uniform size distribution and display physical stability for extended periods of time of more than one year.

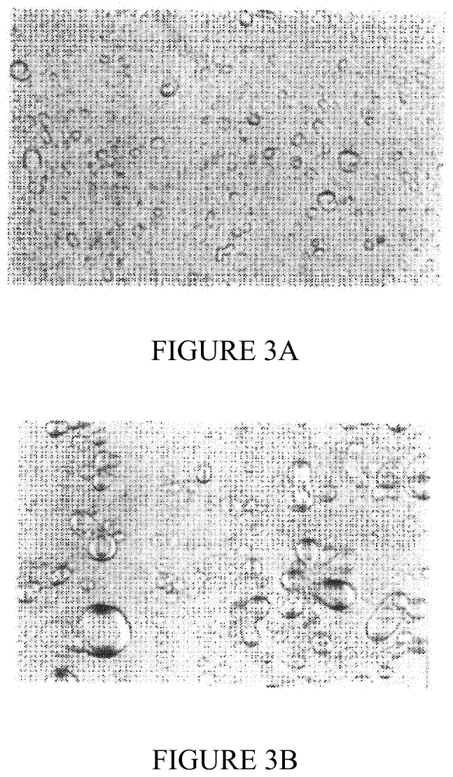

In order to demonstrate the difference in properties observed in the liposome population produced in accordance with two methods, are comparative tests between two liposome compositions prepared from the same ingredients but using in one case the solvent evaporation method and in the other case an anhydrous plastic proliposome gel method. FIG. 3A is a scanned image of the liposome population prepared using the anhydrous proliposome gel (‘melt’ or ‘fusion’) method and FIG. 3B is a scanned image of the liposome population prepared using the solvent evaporation method. As can be seen, the liposome population obtained using the anhydrous plastic proliposome gel method has a liposome size distribution which is substantially more uniform than that obtained using the solvent evaporation method. Also, minimal amounts of aggregated or fused liposomes are formed when using the anhydrous plastic proliposome gel method, whereas large aggregates can be observed in the liposome population obtained using the solvent evaporation method.

In some embodiments of the invention, the lipophilic substance is an oil or solid/semisolid lipophilic consistency enhancer which can be encapsulated into liposomes. As solid or semisolid lipophilic consistency enhancers there are mentioned fatty alcohols, waxes, fatty alcohol fatty acid esters, glyceride esters, white petrolatum and mixtures thereof. Examples of oils which have successfully been encapsulated into liposomes include pentaerythritol tetracaprylate/caprate, pentaerythritol tetraisostearate, cetearyl octanoate and canola oil, jojoba oil, peanut oil, rice bran oil, cottonseed oil, sunflower oil, corn oil, walnut oil, avocado oil, peru balsam, clove oil and eugenol and mixtures thereof. Plant extracts based on oil can also be successfully incorporated into liposomes. Solid/semi solid lipophilic consistency enhancer ingredients can be selected from waxes, fatty alcohols, fatty acid esters, glyceryl stearate, petrolatum or combinations thereof. Specific examples of preferred consistency enhancers include beeswax, glyceryl tribehenate, glyceryl stearate, stearyl heptanoate, stearyl palmitate, cetyl alcohol, stearyl alcohol, myristyl myristate, behenyl erucate and cetyl palmitate and mixtures thereof.

The viscosity of a composition of vesicles in accordance with the invention and containing a consistency enhancer is greater than the viscosity of corresponding vesicles that do not include a consistency enhancer but are otherwise identical. By varying the amount of consistency enhancer it is possible to achieve virtually any required viscosity, from a relatively mobile liquid, to a “lotion”, to “creamy” to “thick cream” to “semi-solid”. The amounts of consistency enhancer required to achieve a particular viscosity of the composition can be determined by routine experiment.

The surfactant used to coat the oil droplet or the solid/semisolid lipophilic consistency enhancer ingredients is important for the successful encapsulation of a lipophilic core into multilamellar lipid vesicles. Primary cationic emulsifiers provide acceptable results. In aspects, the surfactant is benzalkonium chloride or other cationic surfactants such as benzethonium chloride, cetylpyridinium chloride, cetrimide. Nonionic or amphoteric surfactants can also be used, such as naturally derived emulsifiers: PEG-60 almond glycerides, avocado oil diethanolamine, ethoxylated jojoba oil (PEG-40 Jojoba acid and PEG-40 Jojoba alcohol); polyoxyethylene derivatives: polyoxyethylene (20) sorbitan monooleate, polyoxyethylene (20) sorbitan monostearate; lanolin derivatives: polychol 20 (Laneth 20), polychol 40 (laneth 40); neutral phosphate esters: PPG-cetyl ether phosphate, DEA oleth-3 phosphate. It is also possible to use anionic surfactants such as acylglutamates: TEA-cocoyl glutamate, sodium lauroyl glutamate, sodium hydrogenated tallow glutamate and sodium cocoyl glutamate. It is desirable that the surfactant has a high critical micellar concentration (CMC).

In an aspect, the composition described herein is a MLV delivery system for a cannabinoid comprising surfactant in admixture with a cannabinoid in a topical formulation, wherein, the MLV delivery system, when in contact with the skin or mucosal membrane, releases the cannabinoid in a therapeutically-effective amount to provide a localized or systemic effect for treatment of pain or pain-associated condition.

When preparing the lipophilic cannabinoid-in-water emulsion, the hydrophilic ingredients and surfactants are all incorporated in water. Once the water phase of the emulsion has been prepared, the oil and/or solid/semisolid lipophilic ingredients and cannabinoid are added to the water in a homogenizer for a period of time ranging from 5 to 30 minutes to obtain relatively small droplet size. In aspects droplet size ranges from 0.1 μm to 1 μm, in further aspects below about 0.5 μm. The lipid phase melt (anhydrous plastic proliposome gel) is then heated and the lipophilic cannabinoid-in-water emulsion is added and vigorously mixed by either vortexing or propeller mixing depending on the product size.

The composition/formulation procedure is adaptable for large scale manufacturing. The propeller mixing approach can be directly scaled up by geometrically increasing the size of the vessel and the diameter of the propeller mixer. However, as the vessel size increases, a preferred set up might be a combination mixer such as a high intensity mixer with propeller mixer and a scraped surface agitator. In a large scale operation, the lipophilic substance (called the oil phase)-in-water emulsion can be pumped from a first tank into a second tank containing the anhydrous plastic pro-liposome gel at the required temperature and mixed.

With the multi-lamellar lipid vesicle of the present invention, oil droplets containing solubilized cannabinoid can be delivered through liposome encapsulation. Furthermore, the possibility of multi-compartment encapsulation provides cannabinoid release over extended periods of time. Also, encapsulation of lipophilic solid/semisolid consistency enhancers into the central lipophilic core compartment provides enhanced viscosity to the final liposome composition. In this case, the addition of viscosity-increasing agents in the final liposome preparation can be avoided. Overall, the preparation of multi-lamellar lipid vesicles with a central emulsion core component provides a physically stable, uniform liposome composition. The composition has a viscosity that is suitable for topical and transdermal administration and can be easily manufactured on a large scale.

Without being limited to any theory, it is believed that the biphasix nature of the cannabinoid composition provides for both topical treatment of the mucosal layer as well as penetration of the vesicles into the mucosal layer and endocytosis to gain access to the intracellular space. Binary treatment of the mucosal layer is achieved by the biphasix nature of the composition which allows the extra-vesicular emulsion to target the topical mucosal layer while the vesicles can penetrate into the lipophilic mucosa and promote endocytosis which will result in vesicle rupture.

In addition, the biphasix nature of the composition and the oil-in-water emulsion used permits one of skill in the art to provide for a cream or lotion with a viscosity such will be retained at the point of application for a sufficient period of time to allow therapeutic release of the cannabinoid.

### Example Two: Manufacturing Process for the Formulation

### Step 1. Preparation of Oil-in-Water Submicron Emulsion:

Olive oil, glycerol monostearate 40-55 Type I, cetyl alcohol and butylated hydroxy toluene are melted together at 75° C.+/−5° C. The aqueous component of the emulsion including purified water, PEG-40 castor oil hydrogenated, benzalkonium chloride 50% solution, methylparaben, propylparaben, L-methionine, edetate disodium dihydrate, and phosphates are heated together in a stainless steel vessel at 75° C.+5° C. while stirring until the ingredients are dissolved. The oil component (75° C.+/−5° C.) is then added to the aqueous component (75° C.+/−5° C.) gradually, while mixing to form a coarse emulsion. Coarse emulsion is then homogenized by processing through a Microfluidizer until a homogeneous emulsion is formed. This submicron emulsion is cooled down to 8° C.-12° C.

### Step 2: Preparation of the Lipid Phase:

The lipid phase is prepared by melting Phospholipon 90H, cholesterol and butylated hydroxy toluene with propylene glycol in a mixer by heating to about 80-90° C. while mixing at a slow speed. The mixing and heating of the lipid phase ingredients is continued until a clear melt is formed which is then cooled to about 60° C. The required quantity of cannabinoid, for example, CBD, is added and mixed to the lipid phase.

### Step 3: Preparation of the Aqueous Phase:

A mixture of L-methionine, glycine, L-arginine hydrochloride and purified water is gently mixed.

### Step 4: Preparation of CBD MLV Formulation:

The aqueous phase is added to the emulsion from Step 1 in a stainless steel jacketed mixing tank. This mixture is maintained between 8° C.-12° C. while the mixture is mixed slowly and purged with nitrogen gas. The cooled mixture of the emulsion-aqueous phase is rapidly added to the lipid phase which is being mixed at high speed in the mixer. Mixing proceeds for 10-15 minutes while the temperature of the mixture is maintained about 57-60° C. The bulk product thus formed is slowly mixed and cooled to 19° C.-25° C. in a mixer. The product is transferred from the mixer into a stainless steel storage vessel and purged with nitrogen gas. The bulk product is filled into 5 g polypropylene tubes or polypropylene pre-fill applicators. The tubes or applicators are purged with nitrogen and then the required amount of the product is filled into the tubes or pre-fill applicators, which are thermally sealed in case of tubes whereas prefilled applicators are capped. The filled tubes or pre-filled applicators of cannabinoid cream product are stored at 5° C.+/−3° C.

### Example Three: Exemplary Non-Limiting Cannabinoid Formulations for Topical Use

TABLE 1 lists components for a comparative composition lacking an anti-aggregating stabilizing agent such as arginine in a lipid-bilayer composition where the amount of each component is expressed in units of mg/g final composition, and given in both ranges and exemplary quantities (parentheses).

TABLE 1

| | Quantity | |
|---|---|---|
| Component | mg/g | |
| Active Cannabinoid | 0.01-1 to 5 | (0.808) |
| Excipients and protective agents | 1-10 | (2) |
| Benzalkonium Chloride 50% Solution | | |
| Butylated Hydroxytoluene | 0.1-0.5 | (0.102) |
| Cetyl Alcohol | 2-40 | (20.514) |
| Cholesterol | 2-40 | (20) |
| Edetate Disodium Dihydrate | 0.1-0.5 | (0.103) |
| Glycerol Monostearate 40-55, Type 1 | 5-50 | (30.771) |
| Glycine | 0.1-5 | (1) |
| L-Methionine | 0.1-5 | (1.126) |
| Methylparaben | 0.1-5 | (1.538) |
| Olive Oil, Super Refined | 10-70 | (51.285) |
| PEG-40 Castor Oil, Hydrogenated | 10-70 | (51.285) |
| Sodium phosphate, Dibasic, | 1-2 | (1.670) |
| Heptahydrate | | |
| Sodium phosphate, Monobasic, | 0.25-1 | (0.480) |
| anhydrous | | |
| Phospholipon 90H | 60-200 | (100) |
| Propylene Glycol | 30-100 | (69.95) |
| Propylparaben | 0.1-1 | (0.513) |
| Purified Water | Q.S. to 1000 | (646.846) |

TABLE 2 lists components in one exemplary lipid-bilayer composition formed in accordance with the invention, where the amount of each component is expressed in units of mg/g as both ranges and exemplary quantities.

TABLE 2

| | Range | Exemplary quantity |
|---|---|---|
| Excipients | (mg/g) | (mg/g) |
| PEG-40 Castor Oil, Hydrogenated, USP/NF | 10-70 | 51.285 |
| Benzalkonium chloride 50% solution, NF | 1-10 | 2.00 |
| Methylparaben, NF | 0.1-5 | 1.538 |
| Propylparaben, NF | 0.1-1 | 0.513 |
| L-methionine, USP | 0.1-5 | 1.126 |
| Edetate Sodium, dihydrate, USP | 0.1-0.5 | 0.103 |
| Phosphate buffer (composed of Sodium 1-70 | 51.285 | |
| phosphate dibasic heptahydrate USP and | | |
| Sodium phosphate Monobasic USP, anhydrous) | | |
| Purified water, USP | Q.S. to 1000 | 596.72 |
| Olive oil, Super refined, NF | 10-70 | 51.285 |
| Glycerol monostearate 40-55, Type 1, EP | 5-50 | 30.771 |
| Cetyl alcohol, NF | 2-40 | 20.514 |
| Lipid Antioxidant, NF | 0.1-0.5 | 0.102 |
| Phospholipon 90H | 60-200 | 100.00 |
| Cholesterol, NF | 2-40 | 20.00 |
| Propylene glycol, USP | 30-100 | 69.95 |
| Glycine, USP | 0.1-5 | 1.0 |
| L-arginine hydrochloride, USP | 0.1-5 | 1.0 |
| Nitrogen, NF | 0 to Q.S. | n/a |
| Cannabinoid | 0.01-1 to 5 | 2 MIU/g |

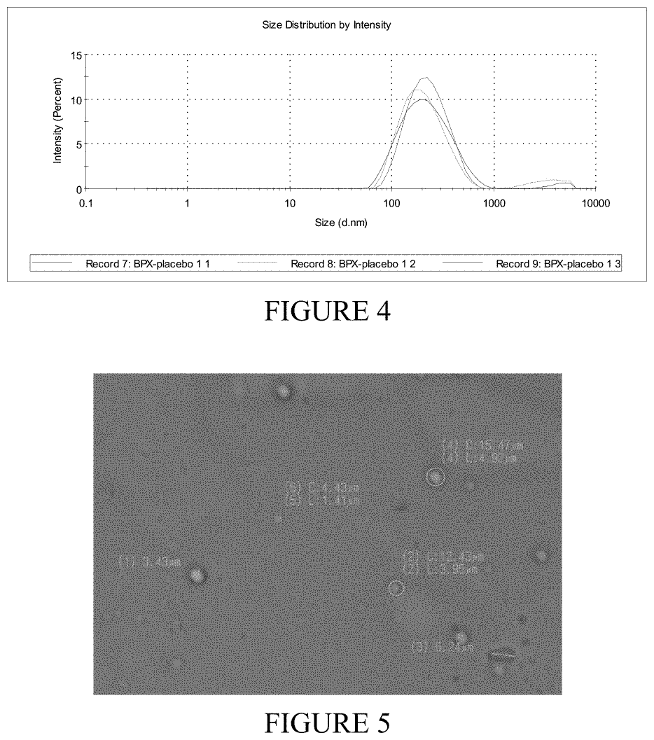

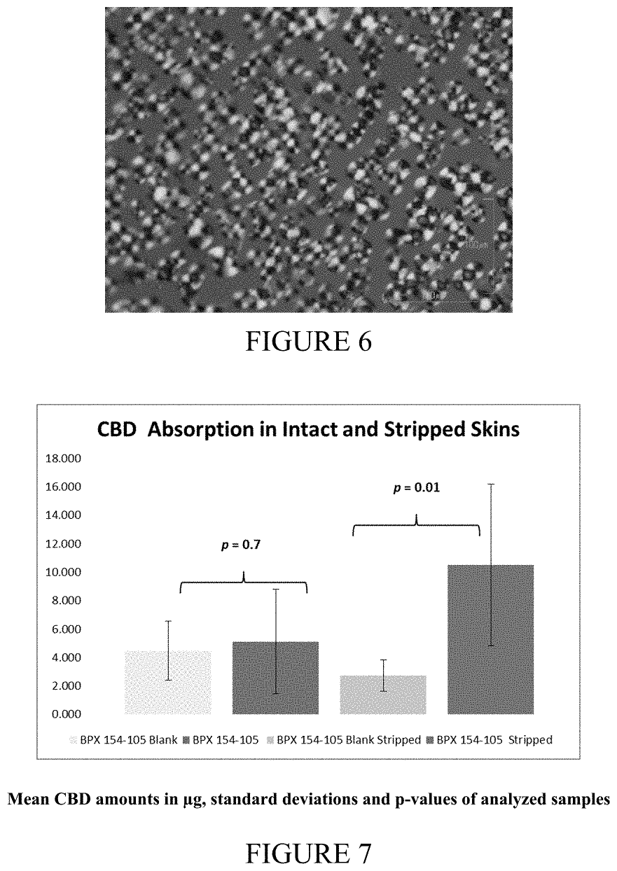

TABLE 3 lists components of a biphasix MVL composition prepared with no CBD (placebo). Particle size distribution pattern of this composition is shown in FIG. 4. FIG. 5 shown an optical microscope image of biphasix placebo oil-in-water emulsion. FIG. 6 shows a 200× magnification of FIG. 4.

TABLE 3

| Composition | Quantity (g) | Preparation method | Results |
|---|---|---|---|
| STEP 1. Emulsion preparation | | | |
| CBD (powder/Oil)-Active | 0 | | |
| Excipients and protective agents | | All ingredients were | Clear liquid |
| Oil phase | | heated up to 75° C. | |
| GELUCIRE 44/14 | 3.077 | until melted and | |
| CREMER Miglyol 810 | 5.129 | dissolved | |
| GELUCIRE 50/13 | 2.051 | | |
| Butylated Hydroxytoluene | 0.01 | | |
| Aqueous phase | | a) All ingredients | Oil-in-water |
| Benzalkonium Chloride 50% Solution | 0.2 | were heated up to | emulsion with |
| Propylparaben | 0.051 | 75° C. until melted | droplets size 4-6 |
| Sodium phosphate, Dibasic, Heptahydrate | 0.167 | and dissolved. | microns are seen |
| Sodium phosphate, Monobasic, anhydrous | 0.048 | The oil phase was | under optical |
| Edetate Disodium Dihydrate | 0.01 | added to the aqueous | microscope |
| | | phase gradually at | |
| | | 75° C. While mixing | |
| | | a coarse emulsion | |
| | | was formed. | |
| | | b) Emulsion is | Micro-emulsion of |
| Kolliphor EL | 5.129 | homogenized by | less than 200 nm |
| | | processing through | droplets was formed |
| | | sonication (3 cycles) | |
| STEP 2. MLV Preparation | | | |
| Phospholipon 90H | 10 | All ingredients were | Vaseline like product |
| Cholesterol | 2 | heated up to 85° C. | thus formed. The |
| Vit E TPGS | 3 | until melted, cooled | microscope image |
| Propylene Glycol | 6.995 | to 60° C. and mixed | shows the self- |
| Purified Water Q.S. to | 61.63 | with emulsion at high | assembly or four 5 |
| | | speed | microns particles, |
| | | | (MLV) |
| | 100 | | |

TABLE 4 lists components of a biphasix MVL CBD composition.

TABLE 4

| Composition | Quantity (g) | Preparation method | Results |
|---|---|---|---|
| STEP 1. Emulsion preparation | | | |
| CBD (powder/Oil)-Active | 1.0 | All ingredients were | Clear yellowish |
| Excipients and protective agents | | heated up to 75° C. | liquid |
| Oil phase | | until melted and | |
| GELUCIRE 44/14 | 0.3 | dissolved | |
| CREMER Miglyol 810 | 0.5 | | |
| GELUCIRE 50/13 | 0.2 | | |
| Butylated Hydroxytoluene | 0.01 | | |
| Aqueous phase | | a) All ingredients | Oil-in-water |
| Benzalkonium Chloride 50% Solution | 0.02 | were heated up to | emulsion with |
| Propylparaben | 0.051 | 75° C. until melted | droplets size 4-6 |
| Sodium phosphate, Dibasic, Heptahydrate | 0.02 | and dissolved. | microns are seen |
| Sodium phosphate, Monobasic, anhydrous | 0.005 | The oil phase was | under optical |
| Edetate Disodium Dihydrate | 0.001 | added to the aqueous | microscope |
| | | phase gradually at | |
| | | 75° C. While mixing | |
| | | a coarse emulsion | |
| | | was formed. | |
| | | b) Emulsion is | Micro-emulsion of |
| Kolliphor EL | 0.5 | homogenized by | less than 200 nm |
| | | processing through | droplets was formed |
| | | sonication (3 cycles) | |
| STEP 2. MLV Preparation | | | |
| Phospholipon 90H | 1.0 | All ingredients were | Vaseline like product |
| Cholesterol | 0.2 | heated up to 85° C. | thus formed. The |
| Vit E TPGS | 0.3 | until melted, cooled | microscope image |
| Propylene Glycol | 0.7 | to 60° C. and mixed | shows the self- |
| Purified Water Q.S. to | 5.193 | with emulsion at high | assembly or four 5 |
| | | speed | microns particles, |
| | | | probably MLV |
| Total | 10.0 | | 9.7% CBD (HPLC) |

One part of CBD-biphasix MLV composition of Table 4 was heated up to 40-50° C. and mixed with 9 parts of ready-to-use topical cream base in order to achieve 1% CBD formulation ready to package and use for the treatment of pain. It is understood by one of skill in the art that the CBD-biphasix MLV composition as shown in Table 4 can be mixed in different ratios to achieve various concentrations of the CBD in the final formulation ready for consumer use. For example ratios of 1:9, 2:8, 3:7, 4:6, 5:5, 6:4, 7:3, 8:2 and 9:1 can be made.

TABLE 5 lists components of a biphasix multilayered lipid vesicle CBD composition.

TABLE 5

| Composition | Quantity (g) | Preparation method | Results |
|---|---|---|---|
| STEP 1. Emulsion preparation | | | |
| CBD (powder/Oil)-Active | 1.0 | All ingredients were | Clear yellowish |
| Excipients and protective agents | | heated up to 75° C. | liquid |
| Oil phase | | until melted and | |
| GELUCIRE 44/14 | 0.4 | dissolved | |
| CREMER Miglyol 810 | 0.6 | | |
| Butylated Hydroxytoluene | 0.01 | | |
| Aqueous phase | | a) All ingredients | Oil-in-water |
| Benzalkonium Chloride 50% Solution | 0.02 | were heated up to | emulsion with |
| Propylparaben | 0.051 | 75° C. until melted | droplets size 4-6 |
| Sodium phosphate, Dibasic, Heptahydrate | 0.02 | and dissolved. | microns are seen |
| Sodium phosphate, Monobasic, anhydrous | 0.005 | The oil phase was | underoptical |
| Edetate Disodium Dihydrate | 0.001 | added to the aqueous | microscope |
| Kolliphor EL | 0.5 | phase gradually at | |
| | | 75° C. While mixing | |
| | | a coarse emulsion | |
| STEP 2. MLV Preparation | | was formed. | |
| Phospholipon 90H | 1.0 | All ingredients were | Vaseline like product |
| Cholesterol | 0.2 | heated up to 85° C. till | thus formed. |
| Vit E TPGS | 0.3 | melted, cooled to | |
| Propylene Glycol | 0.7 | 60° C. and mixed with | |
| Purified Water Q.S. to | 5.193 | emulsion at high | |
| Total | 10.0 | speed | Xx % CBD (HPLC) |

One part of the CBD-BPX MVL composition was heated up to 40-50° C. and mixed with 9 parts of ready-to-use topical cream base in order to achieve 1% CBD composition. Such a CBD formulation can be provided packaged in a tube in desired doses, for example, 20 ml cream representing for example about 40 doses.

### Example Three: Evaluation of biphasix MLV Formulation in Franz diffusion cells Using Full Thickness Porcine Ear Skins

Franz diffusion experiments have evolved into one of the most important methods for researching transdermal drug administration. For semisolid drug products, in vitro release testing (IVRT) is used to evaluate release properties. An in vitro release rate can reflect the combined effect of several physical and chemical parameters, including solubility and particle size of the active and rheological properties of the dosage form.

The most common IVRT method employs an open chamber design like the Franz diffusion cell system and can be used with a synthetic membrane, a tissue construct, or biological sample, such as cadaver skin. The membrane separates the donor compartment containing the test product from the receptor compartment filled with collection medium. Phosphate Buffered Saline (PBS) tends to the collection medium of first choice, though it may not always satisfy the requirements for a viable IVRT method. Diffusion of the drug from the semisolid product across the membrane is monitored by assay of sequentially collected samples of the receptor medium. At predetermined time points, an aliquot of medium is removed from the receptor compartment for drug content analysis, usually by HPLC. The receptor compartment is topped off with fresh medium after each sampling.

### Abbreviations

MSDS Material Safety Data Sheet
NA Not Applicable
OECD Organization for Economic Co-operation and Development
PBS Phosphate Saline Buffer
RT Room Temperature
TBD To Be Determined

The objective of the study is to determine the permeation rate of 1 test items, Formulation BPX-MVL-CBD. The study will be performed in six replicates over sequential 24-hour periods.

The test system used was a PermeGear Franz Cell system with 9 mm jacketed cells with 5 ml receptor volume (cells catalog number 4G-01-00-09-05) 0.64 square cm area.

### Test Items

| Composition BPX-MVL | Quantity (%) |
|---|---|
| Active Ingredient | |
| CBD (Wax)-91% | 1.02 |
| Excipients and protective agents | |
| GELUCIRE 44/14 | 0.71 |
| CREMER Miglyol 810 | 1.18 |
| GELUCIRE 50/13 | 0.47 |
| Butylated Hydroxytoluene | 0.002 |
| Kolliphor EL | 1.18 |
| Phospholipon 90H | 2.36 |
| Vit E TPGS | 1.18 |
| Propylene Glycol | 1.77 |
| Camosic Oil 1% in MCT | 0.24 |
| Total BPX - Solubest | 10.17 |
| Base cream | 89.83 |
| Ingredients: Water, Ceteraryl octanoate, Glycerin, Shea butter, Sweet | |
| almond oil, Palm oil, Jojoba oil, aloe barbaensis, Maris sal, Potassium | |
| sorbate, Sclerotium gum, Xanthan gum, Tocopheryl acetate, Camellia | |
| sinensis leaf extrat, Corral powder | |
| TOTAL | 100 |

### Chemicals—Receptor Compartment Buffer

### 0.5% Cyclodextrin Solution

0.5% cyclodextrin solution in Dulbecco phosphate buffered saline.

| Material | % w/v | Weight/Volume | |
|---|---|---|---|
| Cyclodextrin | 0.5 | 0.25 | g |
| PBS to | 100 | 50 | ml |

### Materials: Dulbecco's Phosphate Buffered Saline

| | |
|---|---|
| Manufacturer: | Biological industries |
| Catalog No | 02-020-1A |
| Batch No.: | 1649990 |
| Physical State: | Liquid |
| Supplied by: | Biological industries |
| Storage Conditions: | 2-8° C. |
| Name in the report: | PBS |

### Name: Cyclodextrin

| | |
|---|---|
| Manufacturer | Merck |
| Catalog No | 142020 |
| Batch No.: | K45304520 |
| Physical State: | solid |
| Supplied by: | Mercury |
| Storage Conditions: | room temp |
| Name in the report: | Cyclodextrin solution |

### Wash Solution 1

### Name: Dulbecco's Phosphate Buffered Saline

| | |
|---|---|
| Manufacturer: | Biological industries |
| Catalog No.: | 02-020-1A |
| Batch No.: | 1649990 |
| Physical State: | Liquid |
| Supplied by: | Biological industries |
| Storage Conditions: | 2-8° C. |
| Expiry Date: | December 2018 |
| Name in the report: | PBS |

Wash solution 2 and Extraction Solution

### Name: Methanol

| | |
|---|---|
| Manufacturer: | JD Baker |
| Catalog No.: | 9093-68 |
| Batch No.: | 0000164804 |
| Physical State: | Liquid |
| Supplied by: | BeitHaDekel |
| Storage Conditions: | 2-8° C. |
| Expiry Date: | 27 Dec. 2017 |
| Name in the report: | Methanol |

### Formulations

The test formulation is a Biphasix formulation with CBD as the active agent. The composition of -BPX-MVL CBD is described in #4.2

### General Equipment

Franz cells 4G-01-00-09-05 describes a 9 mm jacketed cell with a flat ground (ground o-ring) joint and clear glass with a 5 ml receptor volume. This is the most common variety of Franz Cell made
PermeGear V6B Stirrer
Water pump: Freed Electric TEP-4

### Permeation Experiment—UNSTRIPPED Skin

Refrigerated full thickness porcine ear skin (supplied by Lahav) were delivered to Solubest prior to study initiation and maintained according to the supplier's recommendation. OECD guidelines recommend using the skin within 24 hours from excision.
Each permeation experiment was conducted from a 12 cm*4 cm 200-400 micron thick piece of full thickness porcine ear skin. Skin pieces of 2×2 cm were prepared.
Zero time skin samples were prepared for “background effect” evaluation. For this purpose about 100 mg of the cream formulation were loaded on the 3 skin samples, than cream were removed as soon as possible, skin samples were washed and proceed according to procedures below.
Permeation experiments were carried out on 6 replicate cells each with Formulations -BPX-MVL-CBD.
The receptor compartments contained 0.5% cyclodextrin solution in PBS.
A water thermostat was set to temp 33±1° C. in order to keep fixed temp in receptor department at 32±1° C. The water pump kept water to be circulated during experiment. Room temperature will be recorded with data logger.
The following steps describe the operation of the Franz cells:
   Pipette 5 ml of receptor compartment buffer into the receptor compartment.
   Start receptor cell mixing.
   Pre-warm the receptor compartment buffer to 32° C. in the Franz cell equipment for 30 min.
   Mount the porcine skin. Document the experiment date, the date of skin delivery and skin lot

TABLE 6
Skin Information

| Permiation | Porcine skin | Porcine skin lot or |
|---|---|---|
| 1 Date | delivery date | other information |
| Sep. 9, 2018 | Sep. 4, 2018 | 200-400 microns thickness |

The tissues were equilibrated for 30±15 min to permeation temperature prior to weighting and placing the Test Items. Care should be taken to dislodge any air bubbles trapped beneath the membrane.
The syringes were filled with 200±20 microliter of test Items and each weight was documented in the table 2a and 2b below. About 100 microliters of test Items were placed into the donor compartment skin samples and on time point zero samples. The skin surfaces were covered completely. The weight of each syringe with the remaining was subtracted.

TABLE 7
Zero Time Skins and Cream Weights in mg

| Date/time | ZT-1 skin | ZT-2 skin | ZT-3 skin |
|---|---|---|---|
| Sep. 4, 2018 | Weight | Weight | Weight |
| | 85.13 | 95.85 | 86.56 |
| Date/time | ZT-1 cream | ZT-2 cream | ZT-3 cream |
| Sep. 4, 2018 | weight | weight | weight |
| | 154.02 | 151.07 | 146.13 |

TABLE 8
Donor Compartment Skins and Cream Weights

| Date/time | DC-1 skin | DC -2 skin | DC -3 skin | DC -4 skin | DC -5 skin | DC -6 skin |
|---|---|---|---|---|---|---|
| Sep. 4, 2018 | Weight | Weight | Weight | Weight | Weight | Weigh |
| | 86.64 | 115.75 | 93.47 | 102.68 | 114.94 | 94.93 |
| Date/time | DC-1 cream | DC -2 cream | DC -3 cream | DC -4 cream | DC -5 cream | DC -6 cream |
| Sep. 4, 2018 | weight | weight | weight | weight | weight | weight |
| | 171.33 | 95.32 | 138.69 | 183.52 | 162.70 | 137.59 |

The donor compartments were covered with Parafilm to prevent evaporation
After 24 hours 2-mL samples from each receptor compartment were collected into an Eppendorf tube.
The collected samples were stored at 2-8° C. not more than 4 hours and moved to −20±2° C. until delivery to the analytical department.

TABLE 9
Receptor Fluid Samples Volume (ml)

| | | | | | | |
|---|---|---|---|---|---|---|
| Time | RF-1 | RF -2 | RF- 3 | RF- 4 | RF -5 | RF -6 |
| 24 h | | | | | | |
| Date/time | 2 ml | 2 ml | 2 ml | 2 ml | 2 ml | 2 ml |
| Oct. 4, 2018 | | | | | | |

At the end of the permeation phase of the study, the remaining of the Test Items were carefully cleaned with a swab soaked with phosphate buffer pH 7.4. The porcine skins were thoroughly washed by the Phosphate buffer and methanol according to appendix 1 procedure.
Clean skin pieces were weighed, ground and extracted for 24 hours at RT according to Appendix 1 procedure. The extracts were then stored at −20±2° C. until delivery for analysis.

TABLE 10
Skin Samples Weights after 24 hours

| Date | DC-1-24 | DC -2-24 | DC -3-24 | DC -4-24 | DC -5-24 | DC -6-24 |
|---|---|---|---|---|---|---|
| Oct. 4, 2018 | 66.99 | 87.98 | 76.97 | 81.96 | 87.02 | 72.00 |

No visual damages or holes were observed on the skins removed from the donor compartments in the end of experiments.
Results: All received samples (6 samples from receptor compartment, 3 samples from “zero time”, 6 samples from permeation experiments) were analyzed according to Pivot CBD HPLC bioanalytical method PIVOT-AM-002).

The residual creams from donor compartment were swabbed by cotton sticks from each unit and analyzed according to Pivot CBD HPLC analytical method PIVOT-AM-001. These results were taken for mass balance calculations. The summary of results are reported in table 11.

TABLE 11
CBD concentration in creams placed in donor compartment

| | CBD Concentration in mg | | | | | |
|---|---|---|---|---|---|---|
| Sample | Sample 1 | Sample 2 | Sample 3 | Sample 4 | Sample 5 | Sample 6 |
| Residual | 1.33 | 1.27 | 1.27 | 1.24 | 1.24 | 1.19 |
| cream | | | | | | |

TABLE 12
CBD concentration in skins and receptor

| Sample | Sample 1 | Sample 2 | Sample 3 | Sample 4 | Sample 5 | Sample 6 |
|---|---|---|---|---|---|---|
| Receptor | 0 | 0 | 0 | 0 | 0 | 0 |
| Compartment | | | | | | |
| Skin Sam | 3.1348 | 3.9413 | 1.3290 | 5.9475 | 4.4612 | |
| Extracts | | | | | | |
| Zero time | 2.7290 | 3.9337 | 6.7718 | — | — | — |
| sample | | | | | | |

### APPENDIX I

### Rinsing

Cut a circle around the permeation area, may sure no leave the permeation area intact
Wipe any excess formulation off the skin using clean paper towel
Lightly wipe the skin with a methanol soaked paper towel
Rinse each skin piece by immersing in 10 ml PBS
Rinse using syringe with 50 ml PBS
Dry with paper towel
Weight
If unable to extract the tissue immediately, place in freezer at −20° C.

### Tissue Grinding

Cryogenic Grinding with Mortar & Pestle: Grinding tissue samples frozen with liquid nitrogen using a mortar and pestle is a widely used method. The mortar and pestle are cleaned and placed in a Styrofoam tub or cooler where liquid nitrogen is poured or dispensed onto the mortar and pestle. Care is needed to avoid splattering liquid nitrogen when the mortar and pestle first start chilling. After several minutes the set will be cooled and a fog will usually settle over the apparatus. The sample is snap frozen by dropping it into a beaker of liquid nitrogen (use plastic beakers). To grind, hold the pestle with a gloved hand and firmly press on the sample while twisting. The sample will typically shatter into small pieces, some of which may splatter from the mortar, so extra caution is required when handling biohazardous materials. Let the nitrogen evaporate. Once the grinding is completed, residual sample must be tapped or scraped from the pestle.
The sample must then be transferred into a receiving vessel using a spatula into labeled 2 ml Eppendorf vial.
Clean both mortar and pestle with pure ethanol and perform the grinding with a new sample

### Extraction

Into each Eppendorf vial add 1.5 ml Methanol using a volumetric pipette, and close the vial.
Shake the samples using a vortex shaker for 24 h at ambient temp., at low speed.
Freeze at —20 C until analysed.

### Example Four: Evaluation of biphasix MLV Formulation in Franz diffusion cells Using Stripped Porcine Ear Skins

The objective of the study was to determine the permeation rate of 1 test items, Formulation BPX-MVL. The study was performed in six replicates over sequential 24-hour periods. The same Franz Cells test system was used as in Example Three using full thickness porcine skin ears. The same Biphasix formulation, equipment and materials were used as in Example Three.

### Permeation Experiment—Stripped Skin

Refrigerated full thickness porcine ear skin (supplied by Lahav) were delivered to Solubest prior to study initiation and maintained according to the supplier's recommendation. OECD guidelines recommend using the skin within 24 hours from excision.
The permeation experiment was initiated from a 12 cm*4 cm 200-400 micron thick piece of full thickness porcine ear skin. The skin pieces were stripped 5-10 times with adhesive tape until the upper stratum cornea is damaged or removed. Than skin pieces of 2×2 cm will be prepared.
Zero time skin samples were prepared for “background effect” evaluation. For this purpose about 100 mg of the cream formulation were loaded on the 3 stripped skin samples, than cream were removed as soon as possible, skin samples were washed and proceed according to procedures below.
Permeation experiments with 6 stripped skin samples were carried out on 6 replicate cells each with Formulations BPX-MVL.
The receptor compartments contained 0.5% cyclodextrin solution in PBS.
A water thermostat was set to temp 33±1° C. in order to keep fixed temp in receptor department at 32±1° C. The water pump kept water to be circulated during experiment. Room temperature will be recorded with data logger.
The following steps describe the operation of the Franz cells:
   Pipette 5 ml of receptor compartment buffer into the receptor compartment.
   Start receptor cell mixing.
   Pre-warm the receptor compartment buffer to 32° C. in the Franz cell equipment for 30 min.
   Mount the porcine skin. Document the experiment date, the date of skin delivery and skin lot

TABLE 13
Skin Information

| Permeation | Porcine skin | Porcine skin lot or |
|---|---|---|
| 2 Date | delivery date | other information |
| Oct. 4, 2018 | Sep. 4, 2018 | 200-400 microns thickness |

The tissues were equilibrated for 30±15 min to permeation temperature prior to weighting and placing the Test Items. Care should be taken to dislodge any air bubbles trapped beneath the membrane.
The syringes were filled with 200±20 microliter of test Items and each weight was documented in the table 2a and 2b below. About 100 microliters of test Items were placed into the donor compartment skin samples and on time point zero samples. The skin surfaces were covered completely. The weight of each syringe with the remaining was subtracted.

TABLE 14
Zero Time Skins and Cream Weights in mg

| Date/time | ZT-1 skin | ZT-2 skin | ZT-3 skin |
|---|---|---|---|
| Oct. 4, 2018 | Weight | Weight | Weight |
| | 55.90 | 85.84 | 97.16 |
| Date/time | ZT-1 cream | ZT-2 cream | ZT-3 cream |
| Oct. 4, 2018 | weight | weight | weight |
| | 121.11 | 136.84 | 123.18 |

TABLE 15
Donor Compartment Skins and Cream Weights

| Date/time | DC-1 skin | DC -2 skin | DC -3 skin | DC -4 skin | DC -5 skin | DC -6 skin |
|---|---|---|---|---|---|---|
| Nov. 4, 2018 | Weight | Weight | Weight | Weight | Weight | Weigh |
| | 138.19 | 123.09 | 103.59 | 102.70 | 93.96 | 132.76 |
| Date/time | DC-1 cream | DC -2 cream | DC -3 cream | DC -4 cream | DC -5 cream | DC -6 cream |
| Nov. 4, 2018 | weight | weight | weight | weight | weight | weight |
| | 113.92 | 188.73 | 179.24 | 101.63 | 149.97 | 146.65 |

The donor compartments were covered with Parafilm to prevent evaporation.
After 24 hours 2-mL samples from each receptor compartment were collected into an Eppendorf tube.
The collected samples were stored at 2-8° C. not more than 4 hours and moved to −20±2° C. until delivery to the analytical department.

TABLE 16
Receptor Fluid Samples Volume (ml)

| | | | | | | |
|---|---|---|---|---|---|---|
| Time | RF-1 | RF -2 | RF- 3 | RF- 4 | RF -5 | RF -6 |
| 24 h | | | | | | |
| Date/time | 2 ml | 2 ml | 2 ml | 2 ml | 2 ml | 2 ml |
| Nov. 4, 2018 | | | | | | |

At the end of the permeation phase of the study, the remaining of the Test Items were carefully cleaned with a swab soaked with phosphate buffer pH 7.4. The porcine skins were thoroughly washed by the Phosphate buffer and methanol according to appendix 1 procedure of Example 3.
Clean skin pieces were weighed, ground and extracted for 24 hours at RT according to Appendix 1 procedure. The extracts were then stored at −20±2° C. until delivery for analysis.

TABLE 17
Skin Samples Weights after 24 hours

| Date | DC-1-24 | DC -2-24 | DC -3-24 | DC -4-24 | DC -5-24 | DC -6-24 |
|---|---|---|---|---|---|---|
| Oct. 4, 2018 | 82.39 | 66.68 | 59.90 | 60.93 | 63.26 | 86.02 |

No visual damages or holes were observed on the skins removed from the donor compartments 1, 2, 4 & 6 but small hole was seen in the each of skin samples 3 & 5 in the end of experiments

### Results

All received samples (6 samples from receptor compartment, 3 samples from “zero time”, 6 samples from permeation experiments) were analyzed according to Pivot CBD HPLC bioanalytical method PIVOT-AM-002).

The residual creams from donor compartment were swabbed by cotton sticks from each unit and analyzed according to Pivot CBD HPLC analytical method PIVOT-AM-001. These results were taken for mass balance calculations. The summary of results is reported in the table below.

TABLE 18
CBD concentration in creams placed in donor compartment

| | CBD Concentration in mg | | | | | |
|---|---|---|---|---|---|---|
| Sample | Sample 1 | Sample 2 | Sample 3 | Sample 4 | Sample 5 | Sample 6 |
| Residual | 1.40 | 1.37 | 1.27 | 1.19 | 1.27 | 1.50 |
| cream | | | | | | |

TABLE 19
CBD concentration in skins and receptor

| sample | Sample 1 | Sample 2 | Sample 3 | Sample 4 | Sample 5 | Sample 6 |
|---|---|---|---|---|---|---|
| Receptor | 0 | 0 | NA* | 0 | NA* | 0 |
| compartment | | | | | | |
| Skin sample | 6.4750 | 5.8841 | 12.2374 | 4.6971 | 18.6693 | 15.1084 |
| extracts | | | | | | |
| Zero time | 3.2718 | 3.4366 | 1.4482 | — | — | — |
| sample | | | | | | |

NA*—amount of 20.8817 μg CBD was found in receptor #3 and of 47.8251 μg CBD was found in receptor #5. These amounts are not related to permeability values due to the skin damages (holes) found in the end of experiments.

### Discussion

This discussion and conclusion relates to permeation studies 1 & 2 of Example three and four. The methods, materials, used equipment and result parts are described in the protocol and reports of appropriated studies.

### Mass Balance and Recovery Calculation

The initial cream CBD assay is 1%. Therefore the calculated amount of the active material loaded on the each donor compartment has been found by multiplication of the loaded cream amount to the assay value. No CBD was found in the receptor fluids. The total recovered amount was calculated as the sum of CBD content in the residual cream removed from the donor and extracted from the skin samples.

The mass balance was calculated as the ratio of the total recovered to loaded CBD amounts.

TABLE 20
CBD Mass balance in Permeation experiment 1(example three)

| Samples | 1 | 2 | 3 | 4 | 5 | 6 | AVG |
|---|---|---|---|---|---|---|---|
| Loaded cream amount (mg) | 171.33 | 95.32 | 138.69 | 183.52 | 162.70 | 137.59 | |
| CBD amount in cream(mg) | 1.71 | 0.95 | 1.39 | 1.84 | 1.63 | 1.38 | |
| Recovered amount (mg) | 1.33 | 1.27 | 1.27 | 1.24 | 1.24 | 1.19 | |
| from donor | | | | | | | |
| Recovered amount (mg) | 0.003 | 0.004 | 0.001 | 0.006 | 0.004 | 0.012 | |
| from skin | | | | | | | |
| Total Recovered amount (mg) | 1.333 | 1.274 | 1.271 | 1.246 | 1.244 | 1.202 | |
| Recovery (%) | 77.96 | 134.10 | 91.44 | 67.72 | 76.32 | 87.10 | 89.11 |

Similar calculations were done for the values from permeation study 2. The CBD amount found in the receptors #3 and #5 were taken to the calculation here. The results are reported in the table 2.

TABLE 21
CBD Mass balance in Permeation experiment 2

| Samples | 1 | 2 | 3 | 4 | 5 | 6 | AVG |
|---|---|---|---|---|---|---|---|
| Loaded cream amount (mg) | 113.92 | 188.73 | 179.24 | 101.63 | 149.97 | 146.65 | |
| CBD amount in cream(mg) | 1.14 | 1.89 | 1.79 | 1.02 | 1.50 | 1.47 | |
| Recovered amount (mg) | 1.4 | 1.37 | 1.27 | 1.19 | 1.27 | 1.5 | |
| from donor | | | | | | | |
| Recovered amount (mg) | 0.006 | 0.006 | 0.012 | 0.05 | 0.019 | 0.015 | |
| from skin | | | | | | | |
| Recovered amount (mg) | 0 | 0 | 0.021 | 0 | 0.047 | 0 | |
| from receptor chamber | | | | | | | |
| Total Recovered amount (mg) | 1.406 | 1.376 | 1.303 | 1.24 | 1.336 | 1.515 | |
| Recovery (%) | 123.42 | 72.91 | 72.70 | 122.01 | 89.08 | 103.31 | 97.24 |

As can be seen from the tables 20 and 21, the mean mass balance of the permeation experiment 1 with the intact porcine dermatoded skins is 89.11% and of the experiment 2 with stripped skins is 97.24%. These values demonstrate the high CBD recovery during the studies, which is an evident about good CBD stability in the applied cream and the high quality of the conducted experiments.

### Skin Absorption Calculations

As was shown in the report parts, transdermal permeability of CBD, was not observed in the Pivot Studies 1 & 2. The skin CBD absorption can be estimated by the comparison of “24 hours” and “time zero” skin samples extracts. The last were prepared in order to measure the “blank” effect, where the efficacy of the cream washing procedure was tested. The obtained data for Permeation experiment 1 and 2 are reported in the tables 3 and 4 below.

TABLE 22
Individual, Mean and Standard deviation
values found in Permeation experiment 1

| Sample Name | CBD content, μg |
|---|---|
| PIVOT-BPX 154-105-BLANK SAMPLE-1 | 2.7290 |
| PIVOT-BPX 154-105-BLANK SAMPLE-2 | 3.9337 |
| PIVOT-BPX 154-105-BLANK SAMPLE-3 | 6.7718 |
| BPX 154-105 BLANK MEAN | 4.478 |
| SDEV | 2.076 |
| PIVOT-BPX 154-105-24 H SAMPLE-1 | 3.1348 |
| PIVOT-BPX 154-105-24 H SAMPLE-2 | 3.9413 |
| PIVOT-BPX 154-105-24 H SAMPLE-3 | 1.3290 |
| PIVOT-BPX 154-105-24 H SAMPLE-4 | 5.9475 |
| PIVOT-BPX 154-105-24 H SAMPLE-5 | 4.4612 |
| PIVOT-BPX 154-105-24 H SAMPLE-6 | 11.9558 |
| BPX 154-105 24 H MEAN | 5.128 |
| SDEV | 3.676 |

A comparison of the CBD absorption measurements (in μg) was made between the unstripped blank and 24 Hours skin samples using Students t-test for unpaired variates. For this purpose the stratification of “blank” samples results was made, when each value was duplicated in order to compare the equal arrays. Based on these calculations, the difference between CBD absorption into the pig skin samples after 24 hours and “blank” is 1.15 fold (p=0.7). This difference is not significant, so the found CBD amount can be attributed more to a baseline either to skin absorption.

TABLE 23
Individual, Mean and Standard deviation
values found in Permeation experiment 2

| Sample Name | CBD content, μg |
|---|---|
| PIVOT-BPX 154-105-BLANK STRIPPED | 3.2718 |
| SAMPLE-1 | |
| PIVOT-BPX 154-105-BLANK STRIPPED | 3.4366 |
| SAMPLE-2 | |
| PIVOT-BPX 154-105-BLANK STRIPPED | 1.4482 |
| SAMPLE-3 | |
| BPX 154-105 BLANK STRIPPED MEAN | 2.719 |
| SDEV | 1.104 |
| PIVOT-BPX 154-105-24 H STRIPPED SAMPLE-1 | 6.4750 |
| PIVOT-BPX 154-105-24 H STRIPPED SAMPLE-2 | 5.8841 |
| PIVOT-BPX 154-105-24 H STRIPPED SAMPLE-3 | 12.2374 |
| PIVOT-BPX 154-105-24 H STRIPPED SAMPLE-4 | 4.6971 |
| PIVOT-BPX 154-105-24 H STRIPPED SAMPLE-5 | 18.6693 |
| PIVOT-BPX 154-105-24 H STRIPPED SAMPLE-6 | 15.1084 |
| BPX 154-105 24 H STRIPPED MEAN | 10.512 |
| SDEV | 5.695 |

Based on the similar calculating methods above, the CBD amount absorbed into stripped pig skin samples during 24 hours increased for 3.87 folds (p=0.01) vs stripped “blank”. The graphical representation of obtained results is depicted in the FIG. 7.

Examples three and four present a working model for CBD skin absorption, when the intact pig skin represents the healthy and normal dermal conditions and 5-10 stripped skin is the conditions associated with dermatological diseases/conditions. Skin that has a compromised stratum corneum provides a less effective barrier to topically applied chemicals when compared with normal skin. For example, skin that is impaired due to inflammation, irritation, sensitization or more chronic skin disease, such as psoriasis, is likely to be a less effective barrier to the entry of chemicals into the living epidermis and dermis and even to systemic circulation via the dermal route. Gerritsen et al demonstrated that the single course of healthy volunteer skin stripping is more compatible with the psoriasis model than the repeated tape stripping model (Gerritsen et al, Repeated tape stripping of normal skin: a histological assessment and comparison with events seen in psoriasis; Arch Dermatol Res. 1994; 286(8):455-61).

The Johnson & Johnson Consumer & Personal Care Products research group in collaboration with Dermal Technology Laboratory Ltd, Keele, Staffordshire, UK studied the relationship between Trans-Epidermal Water Loss (TEWL), Electrical Resistance (ER) and Tritiated Water Flux (TWF), markers of skin barrier function in OECD (OECD GUIDELINE FOR THE TESTING OF CHEMICALS; 2004) 428 studies as the function of a step-wise reduction in ER from normal (control) skin following 5, 10, 15 or 20 tape strips. It was shown that an in vitro experimental protocol using 5 tape strips, ER and dermatomed pig skin provided a rapid, robust and reproducible approach equivalent to the 3-4-fold increases in TEWL observed clinically in compromised skin (D. J. Davies et al. Development of an in vitro model for studying the penetration of chemicals through compromised skin; Toxicology in Vitro, 2015, (29) 176-181).

### 1% CBD Biphasix MVL Prepared Cream

The CBD skin absorption and permeability was studied using Franz diffusion cells model and OECD Guideline for the Testing of Chemicals, Skin Absorption in vitro method, 2004.

Two comparative studies applying intact and tape stripped dermatomed pig ear skins were conducted.

High level of CBD recovery was shown: 89.1% and 97.2% for intact and stripped skins correspondingly. These mass balances confirm the high quality of conducted experiments and good CBD stability.

No transdermal permeability was shown during this experiment in both types of skin samples (stripped and unstripped).

No significant increase of CBD absorption (1.15 folds vs blank, p=0.7) was shown for the intact skin. These conditions are related to the healthy, not damaged skin. Mild to moderate massage can help to enhance CBD absorption into intact skin.

Significant increase of CBD absorption (3.87 folds vs blank, p=0.01) was demonstrated for the 5-10 one cycle stripped skin. This approach is considered as in-vitro model, which could replicate the typical changes in barrier function observed in humans with compromised skin.

Topical 1% CBD cream can be beneficial to relief the pain, reduce irritation and inflammation in many compromised skin involving dermatological diseases including but not limited to acne, psoriasis, sun and heat burns. The cream may be beneficial for transdermal treatment when repeatedly used in a vigorous massaging manner. This can be improved with formulations made with a looser consistency such as a lotion.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

Although the invention has been described with respect to particular embodiments, it will be apparent to those skilled in the art that various changes and modifications can be made without departing from the invention.

1. A biphasix multilayered lipid vesicle cannabinoid composition comprising:
(a) a first phase comprising a first oil-in-water emulsion; and
(b) a second phase suspended in the first phase, the second phase comprising multilamellar lipid vesicles, the multilamellar lipid vesicles entrapping a second oil-in-water emulsion,
wherein at least one of the first and second oil-in-water emulsions comprises a therapeutically effective amount of a Cannabis-derived compound.

2. The biphasix multilayered lipid vesicle cannabinoid composition of claim 1, wherein the cannabinoid is a natural or a synthetic cannabinoid selected from the group consisting of tetrahydrocannabinols (THC), Δ9-THC, 9-THC propyl analogue (THC-V); Cannabidiol (CBD); Cannabidiol propyl analogue (CBD-V); Cannabinol (CBN), Cannabichromene (CBC); Cannabinodiol (CBDL); Cannabicyclol (CBL); Cannabichromene propyl analogue (CBC-V); Cannabielsoin (CBE); Cannabitriol (CBT), Cannabigerol (CBG), pharmaceutically acceptable salts of these cannabinoids, and combinations thereof.

3. The biphasix multilayered lipid vesicle cannabinoid composition of claim 1, wherein the cannabinoid is a cannabinoid prodrug or cannabinoid agonist.

4. The biphasix multilayered lipid vesicle cannabinoid composition of claim 2, wherein the cannabinoid is Cannabidiol (CBD).

5. The biphasix multilayered lipid vesicle cannabinoid composition of claim 3, wherein CBD is present in an amount of up to about 10% by weight of the composition.

6. The biphasix multilayered lipid vesicle cannabinoid composition of claim 1, wherein at least one of the first and second oil-in-water emulsion is comprised of oil droplets having a size of from about 0.1 μm to about 1 μm.

7. The biphasix multilayered lipid vesicle cannabinoid composition of claim 1, wherein the first and second oil-water-emulsions are the same or different.

8. The biphasix multilayered lipid vesicle cannabinoid composition of claim 1, wherein at least 30% of said cannabinoid is entrapped within said multilamellar lipid vesicles.

9. The biphasix multilayered lipid vesicle cannabinoid composition of claim 1, wherein the cannabinoid is entrapped between phospholipid bilayers of the multilamellar lipid vesicles.

10. The biphasix multilayered lipid vesicle cannabinoid composition of claim 1, wherein (a) and (b) comprise the therapeutically effective amount of the cannabinoid.

11. The biphasix multilayered lipid vesicle cannabinoid composition of claim 1, wherein said multilamellar lipid vesicles comprise at least 2% by weight cholesterol.

12. The biphasix multilayered lipid vesicle cannabinoid composition of claim 1, further comprising an anti-oxidant and/or a stabilizer in (a) and/or (b).

13. The biphasix multilayered lipid vesicle cannabinoid composition of claim 1, formulated as a cream, lotion, liquid, gel, foam, drops, suppository, ointment, spray or patch.

14. The biphasix multilayered lipid vesicle cannabinoid composition of claim 13, wherein said formulation comprises up to 20% by weight CBD, up to 15% by weight CBD, up to 10% by weight CBD, up to 5% by weight CBD, up to 4% CBD, up to 3% by weight CBD, up to 2% by weight CBD or up to 1% by weight CBD.

15. The biphasix multilayered lipid vesicle cannabinoid composition of claim 14, wherein said CBD is in a format selected from powder, oil, extract, powderized oil and combinations thereof.

16. The biphasix multilayered lipid vesicle cannabinoid composition of claim 15, wherein said powderized CBD oil comprises concentrated Cannabis oil containing the CBD and a starch powder.

17. The biphasix multilayered lipid vesicle cannabinoid composition of claim 15, formulated as a cream comprising up to 20% by weight CBD and a base cream formulation.

18. The biphasix multilayered lipid vesicle cannabinoid composition of claim 17, wherein said base cream formulation comprises at least two ingredients selected from the group consisting of water, ceteraryl octanoate, glycerin, shea butter, sweet almond oil, palm oil, jojoba oil, aloe barbaensis, maris sal, potassium sorbate, sclerotium gum, xanthum gum, tocopheryl acetate, camellia sinensis leaf extract and corral powder, gelucire, cremer miglycol 810, butylated hydroxytoluene, kolliphor EL, Phospholipon 90H, Vit E TPGS, propylene glycol and carnosic oil.

19. The biphasix multilayered lipid vesicle cannabinoid composition of claim 15, further comprising:
(i) an opioid selected from the group consisting of morphine, fentanyl, oxycodone, codeine and combinations thereof; or
(ii) an anti-inflammatory drug selected from the group consisting of salicylic acid, oxyacetic acid, salicylates, propionic acid derivatives, acetic acid derivatives, enolic acid derivatives, fenamic acid derivatives, coxibs, sulphonanilides and mixtures thereof; or
(iii) an antibiotic selected from the group consisting of chloramphenicol, fusidic acid, fluoroquinolones, aminoglycoside, polymycin B sulfate and mixtures thereof; or
(iv) combinations of (i), (ii) and (iii).

20. The biphasix multilayered lipid vesicle cannabinoid composition of claim 14, wherein the composition penetrates mucosa and/or an epidermal layer of compromised or uncompromised skin, and wherein upon application to said mucosa or said epidermal layer of compromised or uncompromised skin, said cannabinoid is released rapidly followed by controlled slow release.

21. The biphasix multilayered lipid vesicle cannabinoid composition of claim 20, wherein the cannabinoid composition is for alleviating pain.
